SEQUENCE LISTING (85 sequences)

SEQ ID NO: 1 - DNA, 350 bp, Zea mays
aaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttccc

SEQ ID NO: 2 - DNA, 40 bp, Zea mays
cgctccaccgttggacttgctccgctgtcggcatccagaa

SEQ ID NO: 3 - DNA, 40 bp, Zea mays
tccgctgtcggcatccagaaattgcgtggcggagcggcag

SEQ ID NO: 4 - DNA, 40 bp, Zea mays
attgcgtggcggagcggcagacgtgagccggcacggcagg

SEQ ID NO: 5 - DNA, 40 bp, Zea mays
acgtgagccggcacggcaggcggcctcctcctcctctcac

SEQ ID NO: 6 - DNA, 60 bp, Zea mays
cgctccaccgttggacttgctccgctgtcggcatccagaaattgcgtggcggagcggcag

SEQ ID NO: 7 - DNA, 60 bp, Zea mays
tccgctgtcggcatccagaaattgcgtggcggagcggcagacgtgagccggcacggcagg

SEQ ID NO: 8 - DNA, 60 bp, Zea mays
attgcgtggcggagcggcagacgtgagccggcacggcaggcggcctcctcctcctctcac

SEQ ID NO: 9 - DNA, 80 bp, Zea mays
cgctccaccgttggacttgctccgctgtcggcatccagaaattgcgtggcggagcggcagacgtgagccggcacggcagg

SEQ ID NO: 10 - DNA, 80 bp, Zea mays
tccgctgtcggcatccagaaattgcgtggcggagcggcagacgtgagccggcacggcaggcggcctcctcctcctctcac

SEQ ID NO: 11 - DNA, 100 bp, Zea mays
cgctccaccgttggacttgctccgctgtcggcatccagaaattgcgtggcggagcggcagacgtgagccggcacggcaggcggcctcctcctcctctcac

SEQ ID NO: 12 - DNA, 150 bp, Zea mays
cagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcac

SEQ ID NO: 13 - DNA, 200 bp, Zea mays
taacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcac

SEQ ID NO: 14 - DNA, 250 bp, Zea mays
ttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcac

SEQ ID NO: 15 - DNA, 300 bp, Zea mays
aaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcac

SEQ ID NO: 16 - DNA, 110 bp, Zea mays
cgctccaccgttggacttgctccgctgtcggcatccagaaattgcgtggcggagcggcagacgtgagccggcacggcaggcggcctcctcctcctctcacggcaccggca

SEQ ID NO: 17 - DNA, 120 bp, Zea mays
cgctccaccgttggacttgctccgctgtcggcatccagaaattgcgtggcggagcggcagacgtgagccggcacggcaggcggcctcctcctcctctcacggcaccggcagctacggggg

SEQ ID NO: 18 - DNA, 130 bp, Zea mays
cgctccaccgttggacttgctccgctgtcggcatccagaaattgcgtggcggagcggcagacgtgagccggcacggcaggcggcctcctcctcctctcacggcaccggcagctacgggggattcctttcc

SEQ ID NO: 19 - DNA, 140 bp, Zea mays
cgctccaccgttggacttgctccgctgtcggcatccagaaattgcgtggcggagcggcagacgtgagccggcacggcaggcggcctcctcctcctctcacggcaccggcagctacgggggattcctttcccaccgctcct

SEQ ID NO: 20 - DNA, 150 bp, Zea mays
cgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttccc

SEQ ID NO: 21 - DNA, 90 bp, Zea mays
acgtgagccggcacggcaggcggcctcctcctcctctcacggcaccggcagctacgggggattcctttcccaccgctccttcgctttccc

SEQ ID NO: 22 - DNA, 110 bp, Zea mays
attgcgtggcggagcggcagacgtgagccggcacggcaggcggcctcctcctcctctcacggcaccggcagctacgggggattcctttcccaccgctccttcgctttccc

SEQ ID NO: 23 - DNA, 80 bp, Zea mays
acgtgagccggcacggcaggcggcctcctcctcctctcacggcaccggcagctacgggggattcctttcccaccgctcct

SEQ ID NO: 24 - DNA, 70 bp, Zea mays
acgtgagccggcacggcaggcggcctcctcctcctctcacggcaccggcagctacgggggattcctttcc

SEQ ID NO: 25 - DNA, 60 bp, Zea mays
acgtgagccggcacggcaggcggcctcctcctcctctcacggcaccggcagctacggggg

SEQ ID NO: 26 - DNA, 50 bp, Zea mays
acgtgagccggcacggcaggcggcctcctcctcctctcacggcaccggca

SEQ ID NO: 27 - DNA, 20 bp, Zea mays
cgctccaccgttggacttgc

SEQ ID NO: 28 - DNA, 20 bp, Zea mays
tccgctgtcggcatccagaa

SEQ ID NO: 29 - DNA, 20 bp, Zea mays
attgcgtggcggagcggcag

SEQ ID NO: 30 - DNA, 20 bp, Zea mays
acgtgagccggcacggcagg

SEQ ID NO: 31 - DNA, 20 bp, Zea mays
cggcctcctcctcctctcac

SEQ ID NO: 32 - DNA, 44 bp, Zea mays
ttcctcgcccgccgtaataaatagacaccccctccacaccctct

SEQ ID NO: 33 - DNA, 10 bp, Artificial Sequence
  CACGTG motif
gccacgtggc

SEQ ID NO: 34 - DNA, 10 bp, Artificial Sequence
  CACGTG motif
tacacgtgta

SEQ ID NO: 35 - DNA, 10 bp, Artificial Sequence
  CACGTG motif
ggcacgtgcc

SEQ ID NO: 36 - DNA, 10 bp, Artificial Sequence
  CACGTG motif
atcacgtgat

SEQ ID NO: 37 - DNA, 10 bp, Artificial Sequence
  CACGTG motif
ctcacgtgag

SEQ ID NO: 38 - DNA, 10 bp, Artificial Sequence
  CACGTG motif
aacacgtgtt

SEQ ID NO: 39 - DNA, 10 bp, Artificial Sequence
  CACGTG motif
tacacgtggc

SEQ ID NO: 40 - DNA, 10 bp, Artificial Sequence
  CACGTG motif
tccacgtggc

SEQ ID NO: 41 - DNA, 10 bp, Artificial Sequence
  CACGTG motif
gtcacgtgcc

SEQ ID NO: 42 - DNA, 10 bp, Artificial Sequence
  CACGTG motif
gccacgtgcc

SEQ ID NO: 43 - DNA, 10 bp, Artificial Sequence
  CACGTG motif
gccacgtgag

SEQ ID NO: 44 - DNA, 10 bp, Artificial Sequence
  CACGTG motif
gacacgtggc

SEQ ID NO: 45 - DNA, 14 bp, Artificial Sequence
  CACGTG motif
gtgacacgtggcag

SEQ ID NO: 46 - DNA, 47 bp, Artificial Sequence
  primer
gtctagaggatccccaaacgtgcagtttatctatctttatacatata

SEQ ID NO: 47 - DNA, 45 bp, Artificial Sequence
  primer
gtctagaggatccccaaacatgaacagttagacatggtctaaagg

SEQ ID NO: 48 - DNA, 49 bp, Artificial Sequence
  primer
gtctagaggatccccaaacaaatagcttcacctatataatacttcatcc

SEQ ID NO: 49 - DNA, 48 bp, Artificial Sequence
  primer
gtctagaggatccccaaacttttattctattttagcctctaaattaag

SEQ ID NO: 50 - DNA, 56 bp, Artificial Sequence
  primer
gtctagaggatccccaaacaaaattaaacaaataccctttaagaaattaaaaaaac

SEQ ID NO: 51 - DNA, 38 bp, Artificial Sequence
  primer
gtctagaggatccccaaactaacggacaccaaccagcg

SEQ ID NO: 52 - DNA, 33 bp, Artificial Sequence
  primer
gtctagaggatccccaaaccgctccaccgttgg

SEQ ID NO: 53 - DNA, 34 bp, Artificial Sequence
  primer
gtctagaggatccccaaacggcaccggcagctac

SEQ ID NO: 54 - DNA, 19 bp, Artificial Sequence
  primer
gtgtacgaacgctagcagc

SEQ ID NO: 55 - DNA, 40 bp, Artificial Sequence
  primer
cagcaagcttgcatgcctcgagtctagaggatccccaaac

SEQ ID NO: 56 - DNA, 38 bp, Artificial Sequence
  primer
tgcctcgagtcccgggatctccactgacgtaagggatg

SEQ ID NO: 57 - DNA, 45 bp, Artificial Sequence
  primer
acttgtgataacttaattaacgtgtcctctccaaatgaaatgaac

SEQ ID NO: 58 - DNA, 38 bp, Artificial Sequence
  primer
tgcctcgagtcccgggagattagccttttcaatttcag

SEQ ID NO: 59 - DNA, 40 bp, Artificial Sequence
  CACGTG motif
ggcacgtgccggcacgtgccggcacgtgccggcacgtgcc

SEQ ID NO: 60 - DNA, 40 bp, Artificial Sequence
  CACGTG motif
gccacgtgccgccacgtgccgccacgtgccgccacgtgcc

SEQ ID NO: 61 - DNA, 44 bp, Artificial Sequence
  primer
cttgcatgcctcgagtcccgggtgatgccaaacatgtccctcac

SEQ ID NO: 62 - DNA, 43 bp, Artificial Sequence
  primer
acttgtgataacttaattaagattgcggcagcaaagagcaagc

SEQ ID NO: 63 - DNA, 13 bp, Artificial Sequence
  primer
gggctgggcgctg

SEQ ID NO: 64 - DNA, 31 bp, Artificial Sequence
  primer
ggttggggaaaaaggaagggttgcaggcttg

SEQ ID NO: 65 - DNA, 31 bp, Artificial Sequence
  primer
cccttcctttttccccaacctcgtgttgttc

SEQ ID NO: 66 - DNA, 29 bp, Artificial Sequence
  primer
gataacttaattaatctagagtcgacctg

SEQ ID NO: 67 - DNA, 140 bp, Artificial Sequence
  enhancer
cgctccaccgttggacttgctccgctgtcggcatccagaaattgcgtggcggagcggcagacgtgagccggcacggcaggcggcctcctcctcctctcacgccacgtgccgccacgtgccgccacgtgccgccacgtgcc

SEQ ID NO: 68 - DNA, 140 bp, Artificial Sequence
  enhancer
gccacgtgccgccacgtgccgccacgtgccgccacgtgcccgctccaccgttggacttgctccgctgtcggcatccagaaattgcgtggcggagcggcagacgtgagccggcacggcaggcggcctcctcctcctctcac

SEQ ID NO: 69 - DNA, 340 bp, Artificial Sequence
  enhancer
aaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacgccacgtgccgccacgtgccgccacgtgccgccacgtgcc

SEQ ID NO: 70 - DNA, 380 bp, Artificial Sequence
  enhancer
aaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacgccacgtgccgccacgtgccgccacgtgccgccacgtgccgccacgtgccgccacgtgccgccacgtgccgccacgtgcc

SEQ ID NO: 71 - DNA, 390 bp, Artificial Sequence
  enhancer
aaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccgccacgtgccgccacgtgccgccacgtgccgccacgtgcc

SEQ ID NO: 72 - DNA, 18 bp, Artificial Sequence
  primer
gctagggctgggcgctgc

SEQ ID NO: 73 - DNA, 30 bp, Artificial Sequence
  primer
ccgtgcctaccacgtgtgaggttataagag

SEQ ID NO: 74 - DNA, 30 bp, Artificial Sequence
  primer
ctcttataacctcacacgtggtaggcacgg

SEQ ID NO: 75 - DNA, 30 bp, Artificial Sequence
  primer
aatctagagtcgacctgcagaagtaacacc

SEQ ID NO: 76 - DNA, 20 bp, Zea mays
ttggacttgctccgctgtcg

SEQ ID NO: 77 - DNA, 20 bp, Zea mays
gcatccagaaattgcgtggc

SEQ ID NO: 78 - DNA, 20 bp, Zea mays
ggagcggcagacgtgagccg

SEQ ID NO: 79 - DNA, 20 bp, Zea mays
gcacggcaggcggcctcctc

SEQ ID NO: 80 - DNA, 130 bp, Artificial Sequence
  enhancer
cgctccaccgttggacttgctccgctgtcggcatccagaaattgcgtggcggagcggcagacgtgagccggcacggcaggcggcctcctcctcctctcacgccacgtgccgccacgtgccgccacgtgcc

SEQ ID NO: 81 - DNA, 120 bp, Artificial Sequence
  enhancer
cgctccaccgttggacttgctccgctgtcggcatccagaaattgcgtggcggagcggcagacgtgagccggcacggcaggcggcctcctcctcctctcacgccacgtgccgccacgtgcc

SEQ ID NO: 82 - DNA, 110 bp, Artificial Sequence
  enhancer
cgctccaccgttggacttgctccgctgtcggcatccagaaattgcgtggcggagcggcagacgtgagccggcacggcaggcggcctcctcctcctctcacgccacgtgcc

SEQ ID NO: 83 - DNA, 60 bp, Artificial Sequence
  enhancer
acgtgagccggcacggcagggccacgtgccgccacgtgccgccacgtgccgccacgtgcc

SEQ ID NO: 84 - DNA, 60 bp, Artificial Sequence
  enhancer
cggcctcctcctcctctcacgccacgtgccgccacgtgccgccacgtgccgccacgtgcc

SEQ ID NO: 85 - DNA, 60 bp, Artificial Sequence
  enhancer
gcacggcaggcggcctcctcgccacgtgccgccacgtgccgccacgtgccgccacgtgcc

(19) United States
(12) Patent Application Publication
(10) Pub. No.: US 2023/0058847 A1
(43) Pub. Date: Feb. 23, 2023

(54) ENHANCER

(71) Applicants: KANEKA CORPORATION, Osaka-shi, Osaka (JP); RIKEN, Wako-shi, Saitama (JP)

(72) Inventors: Noriko ISHIKAWA, Shizuoka (JP); Masakazu KASHIHARA, Shizuoka (JP); Toshihiko KOMARI, Tokyo (JP); Takeshi NAKANO, Saitama (JP)

(73) Assignees: KANEKA CORPORATION, Osaka-shi, Osaka (JP); RIKEN, Wako-shi, Saitama (JP)

(21) Appl. No.: 17/791,564

(22) PCT Filed: Jan. 7, 2021
(86) PCT No.: PCT/JP2021/000355

§ 371 Date: Jul. 8, 2022

(30) Foreign Application Priority Data
Jan. 10, 2020 (JP) 2020-002781

(51) Int. Cl. C12N 15/82

(52) U.S. Cl. CPC C12N 15/8216; C12N 15/8261

(57) ABSTRACT

An object of the present invention is to provide enhancers useful in enhancing the transcription activity of promoters.

(i) A polynucleotide comprising a sequence of at least 20 consecutive nucleotides in the region of nucleotides 201 to 300 in SEQ ID NO: 1; or (ii) a polynucleotide that consists of a nucleotide sequence having at least 90% sequence identify to that of the polynucleotide (i), and has an effect to enhance promoter transcription activity, is used as an enhancer.

### TECHNICAL FIELD

The present invention relates to a novel enhancer, and more particularly to an enhancer that is incorporated in, or operatively linked to, a promoter and thereby is capable of enhancing the transcription activity of the promoter.

### BACKGROUND ART

For the purpose of development of novel industrially-beneficial plant varieties, conventional breeding techniques have been employed, such as cross-breeding in which different varieties of plants are bred and their progenies are screened out, and mutation breeding in which plants are mutagenized. In recent years, genetically engineered plants have been developed by transferring beneficial genes to plants to cause them to exert beneficial functions, and beneficial crop varieties have been developed through modification of gene functions by genome editing. Introduction of foreign genes and control of the expression of endogenous genes are effective means for developing new varieties. In both of the above cases, it is often effective to cause plants to express a gene of interest at high levels. In order to achieve this purpose, it is commonplace, for example, to utilize potent promoters or to enhance promoters of foreign or endogenous genes. Examples of potent promoters include, but are not limited to, plant viral promoters (e.g., cauliflower mosaic virus 35s promoters), and constitutive expression promoters of housekeeping genes (e.g., ubiquitin and actin), etc. Also, one of effective means for enhancing promoters is utilization of enhancers. Enhancers are factors that promote gene transcription during the process of gene expression, and are formed of various cis element sequences (NPL 1). It is known that examples of cis-regulatory elements include those responsive to abiotic stresses, including temperature, light, water, salt, chemicals, and endogenous hormones, and those responsive to biotic stresses, including pathogenic infections and insect damages (NPL 1). The combination of cis element sequences constituting an enhancer is a critical determinant for regulating gene expression patterns. In general, enhancers are commonly located in the vicinity of promoters, and may exert their effect independently of their orientation.

However, there are only a few easily available enhancers whose effects have been proved. In recent years, with advances in analysis techniques using next-generation sequencers and bioinformatics, it has become increasingly possible to incomprehensively predict candidate factors that are located in the vicinity of neighboring genes or in intergenic regions distant from neighboring genes and which are considered as possible enhancers, throughout the whole genomes of plants (NPL 2). For example, it was reported that enhancers have been predicted in Arabidopsis thaliana (NPL 3), Zea mays (NPL 4), or Triticum aestivum (NPL 5). However, these are merely huge numbers of candidate factors, few of which have been verified to be effective as enhancers. At present, only a very limited number of enhancers are available.

In order to verify whether candidate enhancer factors actually function as enhancers, it is common to use the minimal domain (also called “core domain”) of a cauliflower mosaic virus (CaMV) 35S promoter. This promoter is known per se as a potent constitutive expression promoter, but when all other domains than the minimal domain are eliminated, this minimal domain functions as a minimum promoter factor (minimal promoter) with a low expression level. If any candidate factor with an enhancer effect is combined with this minimal promoter of CaMV 35S, the minimal promoter will exhibit potent promoter activity again. By utilizing this mechanism, verification of enhancer activity can be done easily. Examples of such reported minimal promoters include, but are not limited to, G-box motifs (NPLs 6, 7, PTL 1), abscisic acid-responsive elements (NPL 8), combinations of G-box and W-box motifs (NPL 9), heat-shock elements from Glycine max (NPL 10), and a combination of Oryza sativa GCN4 and AACA motifs (NPL 11). Further, it was reported in the field of animal research that there has been an attempt to construct a more effective gene expression system through incorporation of the G-box motif or a heterogeneous enhancer sequence upstream of the human EF-1α promoter known as a high-expression promoter (NPL 12).

In recent years, with a breakthrough in the development of genome editing techniques using sequence-specific nucleases, including zinc-finger nucleases (ZFNs), TAL effector nucleases (TALENs), meganucleases (MNs), and CRISPR/Cas9, it has been possible to introduce mutations into endogenous genes of interest or to make modifications to targeted sequences (NPL 13, NPL 14). During the process of developing a new variety, it is a very effective means of assembling useful genes to provide a desired gene expression profile through modification of the native promoter of a gene of interest employing genetic engineering or genome editing. For example, as an attempt to induce high expression of a gene by modifying the sequence of a weak native promoter, a study was reported on increased expression of a reporter gene in lima beans induced by linking a reporter gene to a G. max glycinin promoter (GmScream3) in which a part of the sequence is replaced with a G-box motif (NPL 7). In contrast, although some reports make mention of cis-elements present in the native promoter regions targeted by genome editing (NPLs 14, 15), there are few studies that have actually made and verified modifications to cis-elements. As attempts of genome editing of promoters by CRISPR/Cas9, there are some reports on insertion of a GOS2 promoter upstream of Zea mays ARGOS8 involved in drought tolerance, or replacement of Z. mays ARGOS8 with a GOS2 promoter (NPL 16), or on CRISPR/Cas9 mutagenesis of the S1CLV3 promoter involved in the fruit size of tomatoes (NPL 17).

In many case, plant hormones affect growth promotion of plants. Brassinosteroids are known as one of plant hormones. Brassinosteroids are a group of compounds with a steroidal backbone. Brassinosteroids have different effects on the growth of plants, including: (i) promoting the elongation and growth of stems, leaves, and roots; (ii) promoting cell division; (iii) promoting differentiation of mesophyll cells into vascular vessels or tracheids; (iv) promoting ethylene synthesis; (v) promoting the germination of seeds; and (vi) providing tolerance to environmental stresses. With a focus being put on such physiological activities of brassinosteroids, different attempts have been made to increase the biomass of plants by transferring a gene involved in the synthesis or signaling of brassinosteroids into plants (NPL 18, PTL 2). These attempts are made using a technique of transfecting plantlets with the cDNA sequence of a gene of interest linked to a constitutively highly expressed promoter, and are different from attempts to regulate gene expression by modifying the genome sequence of an endogenous gene promoter.

### CITATION LIST

### Patent Literatures

PTL 1: U.S. Pat. No. 6,187,996

PTL 2: International Patent Publication No. WO 2016/056650

### Non Patent Literatures

NPL 1: Weber, et al., 2016, Trends in Plant Science 21, 974-987.

NPL 2: Shlyueva, et al., 2014, Nature Reviews Genetics 15, 272.

NPL 3: Zhu, et al., 2015, The Plant Cell 27, 2415-2426.

NPL 4: Oka, et al., 2017, Genome Biol. 18, 137.

NPL 5: Li, et al., 2019, Genome Biol. 20, 139.

NPL 6: Ishige, et al., 1999, The Plant Journal 18, 443-448.

NPL 7: Zhang, et al., 2019, Plant Biotechnology Journal 17, 724-735.

NPL 8: Ono, et al., 1996, Plant Physiology 112, 483-491.

NPL 9: Liu, et al., 2016, Scientific Reports 6, 20881.

NPL 10: Strittmatter, et al., 1987, Proceedings of the National Academy of Sciences of the United States of America 84, 8986-8990.

NPL 11: Yoshihara, et al., 1996, FEBS Lett. 383, 213-218.

NPL 12: Wang, et al., 2018, European Journal of Pharmaceutical Sciences 123, 539-545.

NPL 13: Yin, et al., 2017, Nature Plants 3, 17107.

NPL 14: Pandiarajan, et al., 2018, Plant Science 277, 132-138.

NPL 15: Swinnen, et al., 2016, Trends in Plant Science 21, 506-515.

NPL 16: Shi, et al., 2017, Plant Biotechnology Journal 15, 207-216.

NPL 17: Rodriguez-Leal, et al., 2017, Cell 171, 470-480.e8.

NPL 18: Wu, et al., 2008, The Plant Cell 20, 2130-2145.

### SUMMARY OF INVENTION

### Technical Problem

As described above, in order to breed a new plant variety by inducing high expression of a gene of interest, it is effective to utilize an enhancer suitable for a promoter of the gene of interest. In particular, in the field of development of new Z. mays or G. max varieties by gene assembly, which has been actively explored, there has been a strong demand to utilize such an enhancer. However, only a limited number of factors have ever been demonstrated to function as an enhancer, and moreover, not all of them are effective in increasing the expression of a gene of interest. In order to increase gene expression, it is important to enhance the transcription activity of a promoter. Therefore, an object of the present invention is to provide enhancers useful in enhancing the transcription activity of promoters.

### Solution to Problem

The present inventors have conducted intensive studies to achieve the aforementioned object, and as a result, found novel enhancers capable of enhancing the transcription activity of a cauliflower mosaic virus (CaMV) 35S minimal promoter by being linked to said promoter. The inventors also found that such novel enhancers not only have an effect to enhance the transcription activity of said promoter, but also are capable of inducing high expression of Zea mays BIL 7 (ZmBIL7) by being incorporated in a ZmBIL7 promoter. Based on these findings, the inventors have completed the present invention.

The present invention is preferably practiced according to the embodiments described below, but is not limited to these embodiments.

### Embodiment 1

An enhancer comprising the following polynucleotide (i) or (ii):
(i) a polynucleotide comprising a sequence of at least 20 consecutive nucleotides in the region of nucleotides 201 to 300 in SEQ ID NO: 1; or
(ii) a polynucleotide that consists of a nucleotide sequence having at least 90% sequence identify to that of the polynucleotide (i), and has an effect to enhance promoter transcription activity.

### Embodiment 2

The enhancer according to Embodiment 1, wherein the polynucleotide (i) comprises a sequence of at least 40 consecutive nucleotides in the region of nucleotides 201 to 300 in SEQ ID NO: 1.

### Embodiment 3

The enhancer according to Embodiment 1 or 2, wherein the polynucleotide (i) comprises a sequence of at least 60 consecutive nucleotides in the region of nucleotides 201 to 300 in SEQ ID NO: 1.

### Embodiment 4

The enhancer according to any one of Embodiments 1 to 3, wherein the polynucleotide (i) comprises a sequence of at least 80 consecutive nucleotides in the region of nucleotides 201 to 300 in SEQ ID NO: 1.

### Embodiment 5

The enhancer according to any one of Embodiments 1 to 4, wherein the polynucleotide (i) comprises a nucleotide sequence represented by the region of nucleotides 261 to 280 in SEQ ID NO: 1, a nucleotide sequence represented by the region of nucleotides 271 to 290 in SEQ ID NO: 1, or a nucleotide sequence represented by the region of nucleotides 281 to 300 in SEQ ID NO: 1.

### Embodiment 6

The enhancer according to any one of Embodiments 1 to 5, wherein the polynucleotide (i) comprises a nucleotide sequence represented by the region of nucleotides 201 to 240 in SEQ ID NO: 1, a nucleotide sequence represented by the region of nucleotides 221 to 260 in SEQ ID NO: 1, a nucleotide sequence represented by the region of nucleotides 241 to 280 in SEQ ID NO: 1, or a nucleotide sequence represented by the region of nucleotides 261 to 300 in SEQ ID NO: 1.

### Embodiment 7

The enhancer according to any one of Embodiments 1 to 6, wherein the polynucleotide (i) comprises a nucleotide sequence represented by the region of nucleotides 201 to 300 in SEQ ID NO: 1.

### Embodiment 8

The enhancer according to any one of Embodiments 1 to 7, wherein the polynucleotide (i) comprises a nucleotide sequence represented by the region of nucleotides 1 to 300 in SEQ ID NO: 1.

### Embodiment 9

The enhancer according to any one of Embodiments 1 to 8, wherein the polynucleotide (i) comprises a nucleotide sequence represented by SEQ ID NO: 1.

### Embodiment 10

The enhancer according to any one of Embodiments 1 to 9, further comprising a nucleic acid fragment having a nucleotide sequence represented by CACGTG, wherein the nucleic acid fragment is operably linked to the polypeptide (i) or (ii).

### Embodiment 11

The enhancer according to Embodiment 10, wherein the enhancer comprises one to ten nucleic acid fragments as recited in Embodiment 10.

### Embodiment 12

The enhancer according to Embodiment 10 or 11, wherein the nucleic acid fragment consists of from 6 to 14 nucleotides.

### Embodiment 13

A nucleic acid construct comprising the enhancer according to any one of Embodiments 1 to 12, and a promoter.

### Embodiment 14

The nucleic acid construct according to Embodiment 13, wherein the enhancer is operably inserted into, or operably linked to, the promoter.

### Embodiment 15

A vector comprising the enhancer according to any one of Embodiments 1 to 12.

### Embodiment 16

A vector comprising the nucleic acid construct according to Embodiment 13 or 14.

### Embodiment 17

A host cell comprising the enhancer according to any one of Embodiments 1 to 12.

### Embodiment 18

A plant transfected with the enhancer according to any one of Embodiments 1 to 12.

### Embodiment 19

A method for enhancing the transcription activity of a promoter, the method comprising a step of operably inserting the enhancer according to any one of Embodiments 1 to 12 into the promoter, or operably linking said enhancer to the promoter.

### Embodiment 20

A method for increasing gene expression, the method comprising the steps of: operably inserting the enhancer according to any one of Embodiments 1 to 12 into the promoter, or operably linking said enhancer to the promoter; and causing expression of a gene located downstream of the promoter.

### Embodiment 21

A method for creating a highly productive plant, the method comprising the steps of: incorporating a nucleic acid molecule in a plant cell, wherein the nucleic acid molecule comprises the enhancer according to any one of Embodiments 1 to 12, and a promoter into which said enhancer is operably inserted or to which said enhancer is operably linked, and has a gene located downstream of the promoter; and creating a plant from the plant cell incorporating the nucleic acid molecule.

### Advantageous Effects of Invention

According to the present invention, enhancers useful in enhancing the transcription activity of promoters can be provided. The enhancers of this invention can be used for various types of promoters, such as promoters of plant genes. In one instance, the enhancer of this invention can be used for the native promoter of Zea mays BIL7 gene (ZmBIL7 promoter).

When the enhancers of the present invention are used in plants, the levels of genes expressed by promoters present in plant cells can be further increased. In many cases, ordinary promoters present in plant cells vary in the level of a gene expressed depending on the type of a plant organ, the time of season, or the like. However, by using the enhancers of this invention, the levels of differently expressed genes can be increased so that the expression of the genes is raised as a whole.

### BRIEF DESCRIPTION OF DRAWINGS

FIG. 1 depicts the structure of the pJT3968 vector.

FIG. 2 depicts the 5′-deletion analysis of ZmUbi promoters. FIG. 2A depicts the structures from ZmUbi promoter region to GUS gene in the pJT3968 vector (harboring PZmUbi_900) and in plasmids derived from the pJT3968 vector. FIG. 2B depicts the relative values of the GUS activity of immature Z. mays embryos transfected with different plasmids with respect to the GUS activity of those embryos transfected with PZmUbi_900, which is taken as 100%.

FIG. 3-1 depicts the nucleotide sequence of each of SEQ ID NOs.

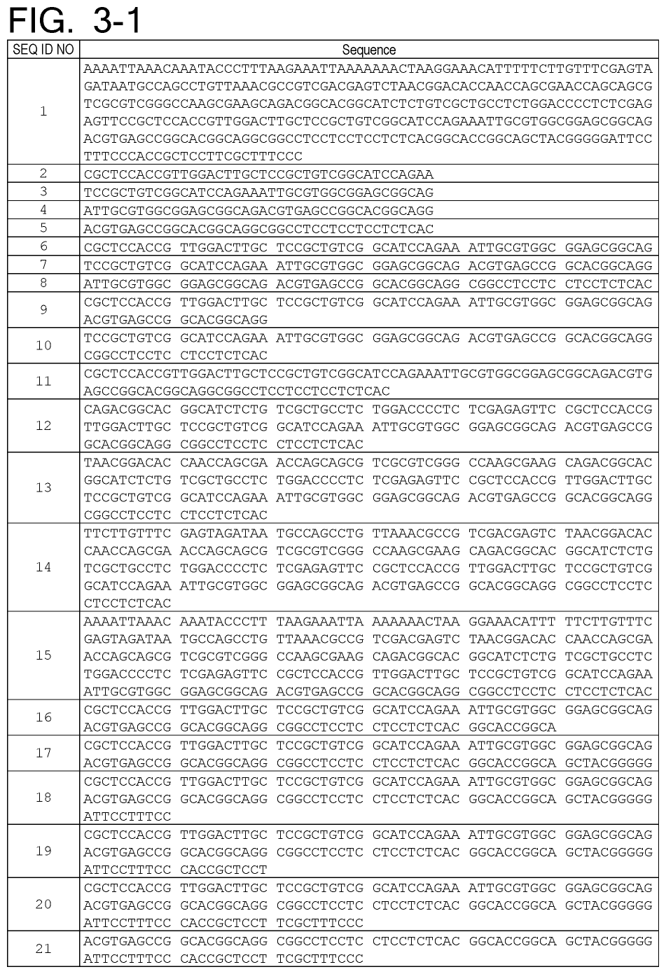

FIG. 3-2 depicts the nucleotide sequence of each of SEQ ID NOs.

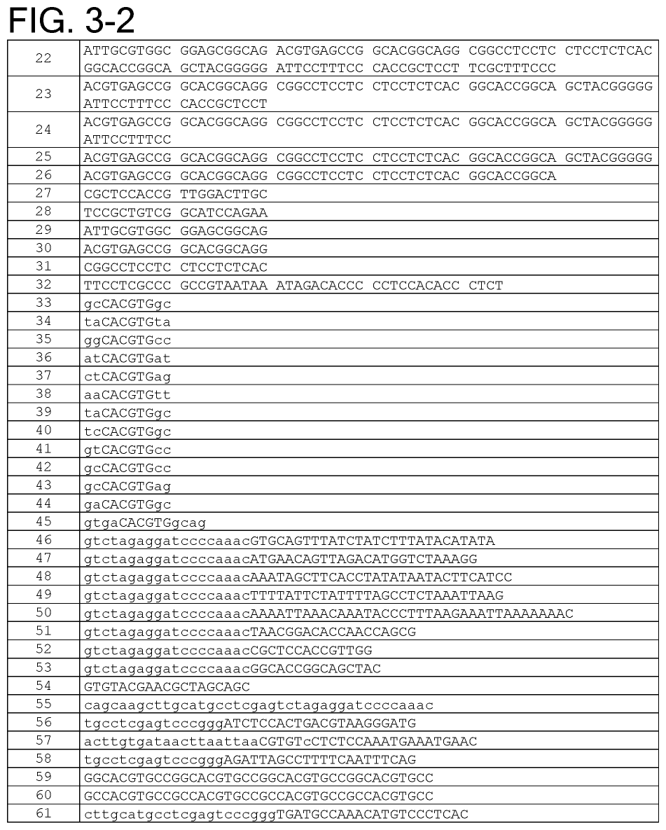

FIG. 3-3 depicts the nucleotide sequence of each of SEQ ID NOs.

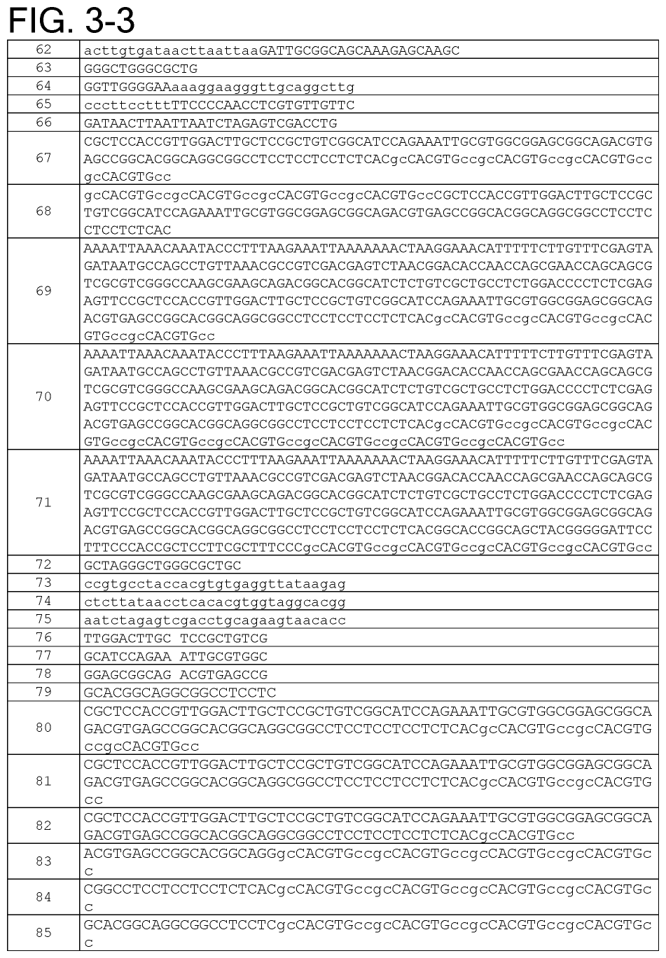

### DESCRIPTION OF EMBODIMENTS

Hereunder, structural features of the present invention will be specifically described, but this invention is not limited thereto. For example, the genetic engineering techniques used in this invention can be practiced according to the known methods described in J. Sambrook, et al. (Molecular Cloning, 2nd ed., Cold Spring Harbor Laboratory Press, 1989; and Molecular Cloning, 3rd ed., Cold Spring Harbor Laboratory Press, 2001).

<Enhancer>

One embodiment of the present invention is directed to an enhancer comprising the following polynucleotide (i) or (ii):
(i) a polynucleotide comprising a sequence of at least 20 consecutive nucleotides in the region of nucleotides 201 to 300 in SEQ ID NO: 1; or
(ii) a polynucleotide that consists of a nucleotide sequence having at least 90% sequence identify to that of the polynucleotide (i), and has an effect to enhance promoter transcription activity.

As referred to herein, the “enhancer” refers to a factor that increases the efficiency of gene transcription driven by a promoter. As referred to herein, the “promoter” refers to a DNA region that determines the transcription start site of a gene and directly modulates the frequency of gene transcription. Gene transcription is initiated when the promoter is bound by a RNA polymerase. In the present invention, the terms “enhancer” and “promoter” differ from each other in a conceptual sense. The enhancer of this invention is capable of enhancing the transcription activity of a promoter, thereby increasing the expression of a gene located downstream of the promoter.

The enhancer of the present invention comprises a polynucleotide, which is characterized by comprising a sequence of at least 20 consecutive nucleotides in the region of nucleotides 201 to 300 in SEQ ID NO: 1 (hereinafter, said polynucleotide is to be referred to as “polynucleotide (i)”). The nucleotide sequence represented by SEQ ID NO: 1 is a nucleotide sequence that indicates a partial region of the Zea mays ubiquitin 1 gene promoter, and is denoted by 350 nucleotides. As referred to herein, the phrases “a polynucleotide comprising a nucleotide sequence” and “a polynucleotide comprises a nucleotide sequence” can also be paraphrased as “a polynucleotide having a nucleotide sequence” and “a polynucleotide has a nucleotide sequence”, respectively.

The polynucleotide (i) preferably comprises a sequence of at least 40 consecutive nucleotides in the region of nucleotides 201 to 300 in SEQ ID NO: 1, more preferably a sequence of at least 60 consecutive nucleotides in the region of nucleotides 201 to 300 in SEQ ID NO: 1, still more preferably a sequence of at least 70 consecutive nucleotides in the region of nucleotides 201 to 300 in SEQ ID NO: 1, yet more preferably a sequence of at least 80 consecutive nucleotides in the region of nucleotides 201 to 300 in SEQ ID NO: 1, further more preferably a sequence of at least 90 consecutive nucleotides in the region of nucleotides 201 to 300 in SEQ ID NO: 1.

The location of a sequence of at least 20 consecutive nucleotides in the region of nucleotides 201 to 300 in SEQ ID NO: 1 found in the polynucleotide (i) is not particularly limited. In the present invention, the polynucleotide (i) comprises, for example, a nucleotide sequence represented by the region of nucleotides 201 to 220 in SEQ ID NO: 1 (SEQ ID NO: 27), a nucleotide sequence represented by the region of nucleotides 211 to 230 in SEQ ID NO: 1 (SEQ ID NO: 76), a nucleotide sequence represented by the region of nucleotides 221 to 240 in SEQ ID NO: 1 (SEQ ID NO: 28), a nucleotide sequence represented by the region of nucleotides 231 to 250 in SEQ ID NO: 1 (SEQ ID NO: 77), a nucleotide sequence represented by the region of nucleotides 241 to 260 in SEQ ID NO: 1 (SEQ ID NO: 29), a nucleotide sequence represented by the region of nucleotides 251 to 270 in SEQ ID NO: 1 (SEQ ID NO: 78), a nucleotide sequence represented by the region of nucleotides 261 to 280 in SEQ ID NO: 1 (SEQ ID NO: 30), a nucleotide sequence represented by the region of nucleotides 271 to 290 in SEQ ID NO: 1 (SEQ ID NO: 79), or a nucleotide sequence represented by the region of nucleotides 281 to 300 in SEQ ID NO: 1 (SEQ ID NO: 31). Preferably, the polynucleotide (i) comprises a nucleotide sequence represented by the region of nucleotides 261 to 280 in SEQ ID NO: 1 (SEQ ID NO: 30), a nucleotide sequence represented by the region of nucleotides 271 to 290 in SEQ ID NO: 1 (SEQ ID NO: 79), or a nucleotide sequence represented by the region of nucleotides 281 to 300 in SEQ ID NO: 1 (SEQ ID NO: 31).

The polynucleotide (i) may comprise a nucleotide sequence represented by the region of nucleotides 201 to 240 in SEQ ID NO: 1 (SEQ ID NO: 2), a nucleotide sequence represented by the region of nucleotides 221 to 260 in SEQ ID NO: 1 (SEQ ID NO: 3), a nucleotide sequence represented by the region of nucleotides 241 to 280 in SEQ ID NO: 1 (SEQ ID NO: 4), or a nucleotide sequence represented by the region of nucleotides 261 to 300 in SEQ ID NO: 1, and preferably comprises a nucleotide sequence represented by the region of nucleotides 261 to 300 in SEQ ID NO: 1 (SEQ ID NO: 5).

The polynucleotide (i) may comprise a nucleotide sequence represented by the region of nucleotides 201 to 260 in SEQ ID NO: 1 (SEQ ID NO: 6), a nucleotide sequence represented by the region of nucleotides 221 to 280 in SEQ ID NO: 1 (SEQ ID NO: 7), or a nucleotide sequence represented by the region of nucleotides 241 to 300 in SEQ ID NO: 1 (SEQ ID NO: 8), and preferably comprises a nucleotide sequence represented by the region of nucleotides 241 to 300 in SEQ ID NO: 1 (SEQ ID NO: 8). The polynucleotide (i) may comprise a nucleotide sequence represented by the region of nucleotides 201 to 280 in SEQ ID NO: 1 (SEQ ID NO: 9), or a nucleotide sequence represented by the region of nucleotides 221 to 300 in SEQ ID NO: 1 (SEQ ID NO: 10), and preferably comprises a nucleotide sequence represented by the region of nucleotides 221 to 300 in SEQ ID NO: 1 (SEQ ID NO: 10). More preferably, the polynucleotide (i) comprises a nucleotide sequence represented by the region of nucleotides 201 to 300 in SEQ ID NO: 1 (SEQ ID NO: 11).

In the present invention, as long as the polynucleotide (i) comprises a sequence of at least 20 consecutive nucleotides in the region of nucleotides 201 to 300 in SEQ ID NO: 1, said polynucleotide may further comprise some other region than the region of nucleotides 201 to 300 in SEQ ID NO: 1. For example, the polynucleotide (i) can comprise a nucleotide sequence represented by the region of nucleotides 151 to 300 in SEQ ID NO: 1 (SEQ ID NO: 12), a nucleotide sequence represented by the region of nucleotides 101 to 300 in SEQ ID NO: 1 (SEQ ID NO: 13), a nucleotide sequence represented by the region of nucleotides 51 to 300 in SEQ ID NO: 1 (SEQ ID NO: 14), or a nucleotide sequence represented by the region of nucleotides 1 to 300 in SEQ ID NO: 1 (SEQ ID NO: 15). In another embodiment, the polynucleotide (i) can comprise a nucleotide sequence represented by the region of nucleotides 201 to 300 in SEQ ID NO: 1 (SEQ ID NO: 16), a nucleotide sequence represented by the region of nucleotides 201 to 320 in SEQ ID NO: 1 (SEQ ID NO: 17), a nucleotide sequence represented by the region of nucleotides 201 to 330 in SEQ ID NO: 1 (SEQ ID NO: 18), a nucleotide sequence represented by the region of nucleotides 201 to 340 in SEQ ID NO: 1 (SEQ ID NO: 19), or a nucleotide sequence represented by the region of nucleotides 201 to 350 in SEQ ID NO: 1 (SEQ ID NO: 20). In still another embodiment, the polynucleotide (i) can comprise a nucleotide sequence represented by the region of nucleotides 261 to 350 in SEQ ID NO: 1 (SEQ ID NO: 21), or a nucleotide sequence represented by the region of nucleotides 241 to 350 in SEQ ID NO: 1 (SEQ ID NO: 22). In yet another embodiment, the polynucleotide (i) can comprise a nucleotide sequence represented by the region of nucleotides 261 to 340 in SEQ ID NO: 1 (SEQ ID NO: 23), a nucleotide sequence represented by the region of nucleotides 261 to 330 in SEQ ID NO: 1 (SEQ ID NO: 24), a nucleotide sequence represented by the region of nucleotides 261 to 320 in SEQ ID NO: 1 (SEQ ID NO: 25), or a nucleotide sequence represented by the region of nucleotides 261 to 310 in SEQ ID NO: 1 (SEQ ID NO: 26). In the nucleotide sequence contained in the polynucleotide (i), any of the nucleotides 1 to 350 in SEQ ID NO: 1 can be an end point, as long as the polynucleotide (i) comprises sequence of at least 20 consecutive nucleotides in the region of nucleotides 201 to 300 in SEQ ID NO: 1. In this invention, the polynucleotide (i) can comprise a nucleotide sequence represented by SEQ ID NO: 1.

The enhancer of the present invention may comprise a polynucleotide that consists of a nucleotide sequence having at least 90% sequence identify to that of the polynucleotide (i) as described above, and has an effect to enhance promoter transcription activity (hereinafter, said polynucleotide is to be referred to as “polynucleotide (ii)”).

The polynucleotide (ii) consists of a nucleotide sequence having at least 90% sequence identify to that of the polynucleotide (i), and preferably consists of a nucleotide sequence having at least 91%, at least 92%, at least 93%, at least 94%, at least 95%, at least 96%, at least 97%, at least 98%, or at least 99% sequence identify to that of the polynucleotide (i).

As referred to herein, the identity in nucleotide sequence refers to an identity in nucleotide sequence between two nucleic acids to be compared, and is expressed by the percentage (%) of identical nucleotides in the optimal alignment of nucleotide sequences made using a mathematical algorithm known in the art. The identity in nucleotide sequence can be determined by visual inspection and mathematical calculation. The identity in nucleotide sequence can also be determined using a computer program. Various sequence comparison computer programs can be used, such as homology search programs (e.g., BLAST, FASTA), sequence alignment programs (e.g., ClustalW)), or genetic information processing software (e.g., GENETYX®), which are well known to those skilled in the art. The identity in nucleotide sequence, as referred to herein, can be specifically determined using an analysis program publicly available on the website of JSpiecies (http://imedea.uib-csic.es/jspecies/) (JSpiecies program based on BLAST software (Richter, M., and Rossello-Mora, R. 2009. Proc. Natl. Acad. Sci. USA 106: 19126-19131)) in its default configuration.

The polynucleotide (ii) is characterized by having an effect to enhance promoter transcription activity. In the present invention, the effect to enhance promoter transcription activity can be examined by the expression level of a gene located downstream of a promoter. In other words, when the expression level of a gene located downstream of a promoter while the polynucleotide (ii) is liked to (or inserted into) the promoter is larger than that while (under the same conditions except that) the polynucleotide (ii) is not linked to (or inserted into) the promoter, it can be determined that the polynucleotide (ii) has an effect to enhance promoter transcription activity. Also, the effect of the enhancer of this invention to enhance promoter transcription activity can be examined in the same way as described above. In other words, when the expression level of a gene located downstream of a promoter while the enhancer of this invention is liked to (or inserted into) the promoter is larger than that while (under the same conditions except that) the enhancer of this invention is not linked to (or inserted into) the promoter, it can be determined that the enhancer of this invention has an effect to enhance promoter transcription activity. The type of a promoter used to evaluate an effect to enhance promoter transcription activity is not particularly limited—for example, a cauliflower mosaic virus (CaMV) 35S minimal promoter or the like can be used.

In the enhancer of the present invention, a plurality of such polynucleotides as described above may be present. For example, one to five such polynucleotides as described above may be present in the enhancer of this invention.

The polynucleotide contained in the enhancer of the present invention may be double-stranded or single-stranded, without particular limitation, and is preferably double-stranded. When the polynucleotide is double-stranded, the double-stranded polynucleotide may be composed of a double strand of DNAs, a double strand of RNAs, or a double strand of DNA and RNA, without particular limitation, and is preferably composed of a double strand of DNAs.

As mentioned above, the enhancer in the present invention differs from a promoter in a conceptual sense. In general, with regard to promoters, it is known that a promoter region about 50 bps upstream of the transcription start site is important for its gene transcription activity. From the viewpoint that the enhancer of this invention differs from a promoter, it is preferred that the enhancer should not comprise a region 44 bps upstream of the transcription start site of the Z. mays ubiquitin 1 gene promoter. In the present description, said region is indicated by the nucleotide sequence represented by SEQ ID NO: 32. In other words, it is preferred that the enhancer of this invention should not comprise a polynucleotide consisting of the nucleotide sequence represented by SEQ ID NO: 32. In general, it is possible that any polynucleotide sequence may exhibit transcription activity and function as a promoter depending on the site of its location. Similarly, it is possible that the enhancer of this invention may also function as a promoter independently without being combined with any other promoter. However, promoter capability differs from enhancer capability—the presence of one capability is not suggestive of the presence of the other capability. Even if the enhancer of this invention also exhibits promoter capability, the presence of such a capability has no particular influence on using it as an enhancer.

The enhancer of the present invention may further comprise a nucleic acid fragment having a nucleotide sequence represented by CACGTG. Said nucleic acid fragment is characterized by being operably linked to the polynucleotide (i) or the polynucleotide (ii). By stating “the nucleic acid fragment is operably linked to the polynucleotide (i) or the polynucleotide (ii)”, it is meant that the nucleic acid fragment is linked to the polynucleotide (i) or the polynucleotide (ii) to the extent that the enhancer of this invention can exert its action and effect. The nucleic acid fragment may be directly (adjacently) linked to the polynucleotide (i) or the polynucleotide (ii), or linked via one or two or more nucleotides to the polynucleotide (i) or the polynucleotide (ii). Also, the nucleic acid fragment may be located upstream or downstream of the polynucleotide (i) or the polynucleotide (ii). In this invention, it is preferred that the nucleic acid fragment should be linked downstream of the polynucleotide (i) or the polynucleotide (ii). When the enhancer of this invention further comprises the nucleic acid fragment, the enhancer can achieve a further increase in its effect to enhance promoter transcription activity. As referred to herein, the phrases “a nucleic acid fragment having a nucleotide sequence” and “a nucleic acid fragment has a nucleotide sequence” can also be paraphrased as “a nucleic acid fragment comprising a nucleotide sequence” and “a nucleic acid fragment comprises a nucleotide sequence”, respectively.

The number of the nucleic acid fragment(s) contained in the enhancer of the present invention is not particularly limited, and is for example, in the range of from 1 to 10, preferably from 2 to 9, more preferably from 3 to 8. When the number of the nucleic acid fragment(s) is 2 or more, said nucleic acid fragments may be directly (adjacently) linked to each other, or linked via one or two or more nucleotides to each other. When the number of the nucleic acid fragment(s) is 2 or more, all of said nucleic acid fragments may be located upstream or downstream of the polynucleotide (i) or the polynucleotide (ii), or some of said nucleic acid fragments may be located upstream of the polynucleotide (i) or the polynucleotide (ii) and the others may be located downstream of the polynucleotide (i) or the polynucleotide (ii). In this invention, it is preferred that when the number of the nucleic acid fragment(s) is 2 or more, all of said nucleic acid fragments should be located downstream of the polynucleotide (i) or the polynucleotide (ii).

The nucleic acid fragment having a nucleotide sequence represented by CACGTG is not particularly limited, and, for example, consists of from 6 to 14 nucleotides, preferably from 8 to 12 nucleotides, more preferably from 9 to 11 nucleotides, most preferably 10 nucleotides. The types of other nucleotides than CACGTG as contained in the nucleic acid fragment are not particularly limited.

In the present invention, the nucleic acid fragment having a nucleotide sequence represented by CACGTG is not particularly limited. Preferably, the nucleic acid fragment having a nucleotide sequence represented by CACGTG is a nucleic acid fragment having a nucleotide sequence represented by SEQ ID NO: 33, a nucleic acid fragment having a nucleotide sequence represented by SEQ ID NO: 34, a nucleic acid fragment having a nucleotide sequence represented by SEQ ID NO: 35, a nucleic acid fragment having a nucleotide sequence represented by SEQ ID NO: 36, a nucleic acid fragment having a nucleotide sequence represented by SEQ ID NO: 37, a nucleic acid fragment having a nucleotide sequence represented by SEQ ID NO: 38, a nucleic acid fragment having a nucleotide sequence represented by SEQ ID NO: 39, a nucleic acid fragment having a nucleotide sequence represented by SEQ ID NO: 40, a nucleic acid fragment having a nucleotide sequence represented by SEQ ID NO: 41, a nucleic acid fragment having a nucleotide sequence represented by SEQ ID NO: 42, a nucleic acid fragment having a nucleotide sequence represented by SEQ ID NO: 43, a nucleic acid fragment having a nucleotide sequence represented by SEQ ID NO: 44, or a nucleic acid fragment having a nucleotide sequence represented by SEQ ID NO: 45. More preferably, the nucleic acid fragment having a nucleotide sequence represented by CACGTG is a nucleic acid fragment having a nucleotide sequence represented by SEQ ID NO: 35, or a nucleic acid fragment having a nucleotide sequence represented by SEQ ID NO: 42. In this invention, only one type of the aforementioned nucleic acid fragments may be used alone, or two or more types of them may be used in combination.

The enhancer of the present invention may further comprise a nucleic acid fragment having a nucleotide sequence containing TTGAC as a common motif, such as a nucleotide sequence represented by TTGAC, TTGACC, TTGACT, TTTGACC, or TTTGACT. Also, the enhancer of this invention may further comprise a nucleic acid fragment having a nucleotide sequence containing GCCCA as a common motif, such as a nucleotide sequence represented by GGCCCA, TGGGCC, AGCCCA, or AGGTGGGCCCGT. Also, the enhancer of this invention may further comprise a nucleic acid fragment having a nucleotide sequence containing GATA as a common motif, such as a nucleotide sequence represented by TGATAG, TGATAA, AGATAG, AGATAA, ATGATAAGG, AAGATAAGATT, or GATAAG. Also, the enhancer of this invention may further comprise a nucleic acid fragment having a nucleotide sequence containing GT as a common motif, such as a nucleotide sequence represented by GGTAATT, GGTAAAT, or GTGTGGTTAATATG. Also, the enhancer of this invention may further comprise a nucleic acid fragment having GAP-box, such as a nucleic acid fragment having a nucleotide sequence represented by CAAATGAAAGA or CAAATGAA. In this invention, only one type of the aforementioned nucleic acid fragments may be used alone, or two or more types of them may be used in combination.

<Nucleic Acid Construct Comprising an Enhancer>

The enhancer of the present invention can be combined with a promoter and used as a nucleic acid construct. In other words, another embodiment of this invention is directed to a nucleic acid construct comprising the enhancer of this invention as described above, and a promoter. Since, as mentioned above, the enhancer of this invention has an effect to enhance promoter transcription activity, the nucleic acid construct comprising the enhancer of this invention and a promoter is capable of increasing the expression level of a gene located downstream of the promoter.

The type of the promoter contained in the nucleic acid construct of the present invention is not particularly limited as long as the promoter is capable of transcribing a gene of interest. However, the promoter is preferably heterogeneous from the enhancer of this invention. In other words, since the enhancer of this invention is derived from the Z. mays ubiquitin 1 gene promoter, the promoter contained in the nucleic acid construct of this invention is preferably a heterogeneous promoter from the Z, mays ubiquitin 1 gene promoter.

In the nucleic acid construct of the present invention, the enhancer may be operably inserted into, or operably linked to, the promoter. By stating herein: “the enhancer is operably inserted into, or operably linked to, the promoter”, it is meant that the enhancer is inserted into, or linked to, the promoter in such a manner that the promoter can exert its function—i.e., the promoter can transcribe a gene of interest.

When the enhancer of the present invention is operably inserted into the promoter, the enhancer may be inserted at the center of the promoter, toward the 5′ end upstream of the center of the promoter, or toward the 3′ end downstream of the center of the promoter—the location of the enhancer is not particularly limited.

When the enhancer of the present invention is operably linked to the promoter, the enhancer may be directly (adjacently) linked to the promoter, or linked via one or two or more nucleotides to the promoter. The location of the enhancer is not particularly limited, but the enhancer is preferably located upstream of the promoter.

The type of the promoter into which the enhancer of this invention is operably inserted, or to which said enhancer is operably linked, is not particularly limited. Examples of the promoter include, but are not limited to, cauliflower mosaic virus 35S promoters (CaMV35S), Zea mays BIL7 gene promoters (ZmBIL7 promoters), different BIL7 gene promoters derived from other specifies than Zea mays, other ubiquitin promoters than the Zea mays ubiquitin 1 gene promoter, different actin promoters, different translation initiation factor promoters, different translation elongation factor promoters, nopaline synthase gene promoters, napin gene promoters, and oleosin gene promoters.

As the promoter into which the enhancer of this invention is operably inserted, or to which said enhancer is operably linked, a promoter having a function of inducing the expression of a nucleic acid in a site-specific manner in a plant can also be used. Examples of such a promoter include, but are not limited to, promoters inducing the expression of a nucleic acid in a leaf-specific manner (e.g., Oryza sativa psbO gene promoters (Japanese Unexamined Patent Application Publication No. JP 2010-166924)), promoters inducing the expression of a nucleic acid in a stem-specific manner (e.g., Arabidopsis thaliana FA6 promoters (Gupta, et al., 2012, The Plant Cell Rep. 31: 839-850.)), promoters inducing the expression of a nucleic acid in a root-specific manner (e.g., RCc3 promoters (Xu, et al., 1995, Plant Mol. Biol. 27: 237-248)), and promoters inducing the expression of a nucleic acid mainly in the vegetable organs of roots, stems or leaves (e.g., Arabidopsis thaliana AS promoters (Wu, et al., 2008, The Plant Cell 20: 2130-2145.)).

Further, as the promoter into which the enhancer of this invention is operably inserted, or to which said enhancer is operably linked, an inducible promoter can be used. Examples of such an inducible promoter include, but are not limited to, promoters known to be induced by external causes, such as fungal, bacterial or viral infections or invasions, low temperatures, high temperatures, drought, UV irradiation, or spray of particular compounds including auxins, brassinosteroids or other hormones. Examples of such promoters include, but are not limited to, Oryza sativa chitinase gene promoters induced by fungal, bacterial, or viral infections or invasions (Xu, et al., 1996, Plant Mol. Biol. 30: 387), tobacco PR protein gene promoters (Ohshima, et al., 1990, The Plant Cell 2: 95), Oryza sativa lip19 gene promoters induced by low temperatures (Aguan, et al., 1993, Mol. Gen. Genet. 240:1), Oryza sativa hsp80 and hsp72 gene promoters induced by high temperatures (Van Breusegem, et al., 1994, Planta 193: 57), Arabidopsis thaliana rab16 gene promoters induced by drought (Mundy, et al., 1990, Proc. Natl. Acad. Sci. USA 87: 1406-1410), parsley chalcone synthase gene promoters induced by UV irradiation (Schulze-Lefert, et al., 1989, EMBO J. 8: 651), Zea mays alcohol dehydrogenase gene promoters induced under anaerobic conditions (Walker, et al., 1987, Proc. Natl. Acad. Sci. USA 84: 6624), and salt stress-induced promoters (Shinozaki and Yamaguchi-Shinozaki., 2000, Curr. Opin. Plant Biol. 3, 217-223).

<Vector>

Both of the enhancer and nucleic acid construct of the present invention as described above can be inserted and used in a vector. In other words, another embodiment of this invention is directed to a vector comprising the enhancer of this invention or the nucleic acid construct of this invention.

A vector can be prepared simply by linking a desired nucleic acid to a recombinant vector available in the art according to a conventional procedure. The vector of this invention is preferably a transformation vector. In particular, when the vector of this invention is intended to be applied to a plant cell, the vector of this invention is preferably a plant transformation vector. The type of a vector used in this invention is not particularly limited, and examples of vectors that can be used include, but are not limited to, pBI vectors, pBluescript vectors, and pUC vectors. Examples of pBI vectors include, but are not limited to, pBI121, pBI101, pBI101.2, pBI101.3, and pBI221. Binary vectors such as pBI vectors are advantageous in that they can be used to transfer a desired nucleic acid to plants through the mediation of Agrobacterium. Examples of pBluescript vectors include, but are not limited to, pBluescript SK(+), pBluescript SK(−), pBluescript II KS(+), pBluescript II KS(−), pBluescript II SK(+), and pBluescript II SK(−). Examples of pUC vectors include, but are not limited to, pUC19 and pUC119. pBluescript and pUC vectors are advantageous in that they can be used to transfer a nucleic acid directly in plants. Further, binary vectors such as pGreen series of vectors (www.pgreen.ac.uk), pCAMBIA series of vectors (www.cambia.org), and pLC series of vectors (International Patent Publication No. WO 2007/148819), and superbinary vectors such as pSB11 (Komari, et al., 1996, Plant J., 10: 165-174), and pSB200 (Komori, et al., 2004, Plant J., 37: 315-325), can be advantageously used.

The vector of the present invention preferably comprises a transcriptional terminator sequence containing a polyadenylation site required for stabilization of a transcript. Those skilled in the art can appropriately select a transcriptional terminator sequence.

The type of a transcriptional terminator sequence is not particularly limited as long as it can function as a transcriptional termination site. Any known transcriptional terminator sequence may be used. The transcriptional terminator sequence can be selected depending on the type of a promoter to be used. For example, the transcriptional termination region of cauliflower mosaic virus 35S (CaMV35S terminator), the transcriptional termination region of nopaline synthase gene (Nos terminator), or the like can be used. By locating the transcriptional terminator sequence at an appropriate position in a recombinant expression vector, the occurrence of a phenomenon in which an unnecessarily long transcript is synthesized after the vector is transferred to a plant cell can be prevented.

The vector of the present invention may further comprise some other nucleic acid segments. The types of such other nucleic acid segments are not particularly limited, and examples thereof include selection markers and nucleotide sequences for enhancing translational efficiency. The vector of this invention may further comprise left (LB) and right (RB) border sequences. These border sequences are required for transfer of T-DNA to a plant cell, particularly when a desired nucleic acid construct in the vector is transfected into a plantlet using Agrobacterium.

As selection markers, drug-resistant genes can be used, for example. Examples of such drug-resistant genes include, but are not limited to, drug-resistant genes for hygromycin, bleomycin, kanamycin, gentamicin, chloramphenicol or the like (e.g., neomycin phosphotransferase genes with resistance to the antibiotics kanamycin and gentamycin, hygromycin phosphotransferase genes with resistance to hygromycin). Phosphinothricin acetyltransferase genes with resistance to the herbicide phosphinothricin can also be used. Thus, by selecting a plantlet growing in a medium containing any of the aforementioned antibiotics and herbicide, a plantlet transfected with a part or the whole of the vector of this invention can be easily sorted out.

Examples of nucleotide sequences for enhancing translational efficiency include, but are not limited to, omega sequence from tobacco mosaic virus. Gene translational efficiency can be improved by locating this omega sequence in the untranslational region (5′ UTR) downstream of a promoter. Also, gene translational efficiency can be enhanced by locating the 5′ UTR of a plant-derived enzyme such as alcohol dehydrogenase downstream of a promoter. As mentioned above, various nucleic acid segments can be incorporated in the vector of this invention depending on the purpose.

The method for constructing the vector of the present invention is not particularly limited. It is only necessary to transfer the enhancer of this invention, a promoter, a gene of interest, a terminator sequence, and optionally other DNA segments into an appropriately selected base vector so as to be arranged in a specified order. Insertion of a nucleic acid into a base vector can be carried out by, for example, a method that involves cleaving a purified nucleic acid with an appropriate restriction enzyme and inserting the cleaved nucleic acid into a restriction enzyme site or multicloning site in an appropriate vector (e.g., Molecular Cloning, 5.61-5.63).

Those skilled in the art can prepare a vector comprising a desired gene, as appropriate, according to a common genetic engineering technique. Such a vector can generally be prepared easily using various types of commercially available vectors.

<Host Cell>

The enhancer of the present invention can be transferred to a cell (host cell). In other words, another embodiment of this invention is directed to a host cell comprising the enhancer of this invention. It is preferred that the enhancer of this invention should be contained exogenously (i.e., as an exogenous substance) in a host cell.

The host cell of the present invention can be an animal cell or a plant cell, without particular limitation. However, in this invention, the host cell is preferably a plant cell. Examples of plant cells include, but are not limited to, various forms of plant cells, such as suspension-cultured cells, protoplasts, and cells in plantlets.

The type of the plant cell is not particularly limited, and cells derived from dicotyledonous plants or monocotyledonous plants can be used. Examples of dicotyledonous plants include, but are not limited to, Arabidopsis thaliana, Glycine max, Gossypium arboretum, Brassica campestris, Beta vulgaris, Nicotiana tabacum, Solanum lycopersicum, Raphanus sativus, Vitis vinifera, and Populus trichocarpa. Among them, A. thaliana, G. max, G. arboretum, B. campestris, N. tabacum, and S. lycopersicum are preferred, with A. thaliana, G. max, G. arboretum, and B. campestris being more preferred. Examples of monocotyledonous plants include, but are not limited to, Oryza sativa, Zea mays, Triticum aestivum, Hordeum vulgare, Sorghum bicolor, Saccharum officinarum, and Allium cepa. Among them, O. sativa, Z. mays, T. aestivum, and Sorghum bicolor are preferred, with O. sativa and Z. mays being more preferred.

The enhancer of the present invention can be transferred to a host cell using the vector of this invention. In this case, the host cell of this invention can be said as a host cell comprising the vector of this invention. A method for inducing high expression of a gene of interest in a host cell can be exemplified by methods in which the gene of interest is incorporated in the vector of this invention and the vector is transferred to a cell by a method known to those skilled in the art, such as polyethylene glycol method, Agrobacterium method, liposome method, cationic liposome method, calcium phosphate precipitation method, electric pulse perforation method (electroporation) (Current Protocols in Molecular Biology, edit. Ausubel, et al., 1987, Publish. John Wiley & Sons., Sections 9.1 to 9.9), lipofection method, microinjection method, or particle gun method. In this invention, the Agrobacterium method can be preferably used. When a nucleic acid is transferred to a plant cell, the nucleic acid may be directly transferred to the plant cell using the microinjection method, the electroporation method, the polyethylene glycol method, or the like, or may be indirectly transferred to the plant cell through a virus or bacterium having an ability to infect a plant, by incorporating the nucleic acid in a plasmid for gene transfer to plants and using the plasmid as a vector. Typical examples of such a virus include, but are not limited to, cauliflower mosaic virus, tobacco mosaic virus, and geminivirus, and examples of such a bacterium include, but are not limited to, Agrobacterium. When gene transfer to plants is performed by the Agrobacterium method, a commercially available plasmid can be used.

Other methods for inducing high expression of a gene of interest in a host cell can be exemplified by methods in which the enhancer of the present invention is transferred to a cell by a method known to those skilled in the art, such as polyethylene glycol method, Agrobacterium method, liposome method, cationic liposome method, calcium phosphate precipitation method, electric pulse perforation method (electroporation) (Current Protocols in Molecular Biology, edit. Ausubel, et al., 1987, Publish. John Wiley & Sons., Sections 9.1 to 9.9), lipofection method, microinjection method, or particle gun method, and then operably inserted into, or operably linked to, a promoter of the gene of interest. When a nucleic acid is transferred to a plant cell, the nucleic acid may be directly transferred to the plant cell using the microinjection method, the electroporation method, the polyethylene glycol method, or the like, or may be indirectly transferred to the plant cell through a virus or bacterium having an ability to infect a plant, by incorporating the nucleic acid in a plasmid for gene transfer to plants and using the plasmid as a vector. Typical examples of such a virus include, but are not limited to, cauliflower mosaic virus, tobacco mosaic virus, and geminivirus, and examples of such a bacterium include, but are not limited to, Agrobacterium. When gene transfer to plants is performed by the Agrobacterium method, a commercially available plasmid can be used.

In order to operably insert the enhancer of the present invention into a promoter of a gene of interest, or to operably link said enhancer to said promoter, a gene targeting technique in which a nucleic acid is inserted into a target site in the genome of a host cell, or a genome editing (also called gene editing) technique can be used. Examples of gene targeting techniques include, but are not limited to, gene targeting with the use of homologous recombination (Terada, et al., 2007, Plant Physiology 144, 846-856). Examples of genome editing techniques include genome editing with the use of enzymes capable of cleaving a target site in the genome of a host cell, such as CRISPR/CAS (Endo, et al., 2016, Plant Physiology 170, 667-677), TALENS (Wang, et al., 2014, Nature Biotechnology 32, 947-951), Zincfinger Nuclease (Duda, et al., 2014, Nucleic Acids Research 42, e84-e84), and Meganuclease (Popplewell, et al., 2013, Human Gene Therapy 24, 692-701). According to those genome editing techniques, when a nucleic acid of interest is transferred to a host cell, any of the aforementioned enzymes or components thereof, or nucleic acids encoding said enzymes or components thereof, is also transferred to the host cell to induce cleavage of a target site, and during the process of repair of the cleavage, the nucleic acid of interest is inserted into the target site (Osakabe and Osakabe, 2015, Plant and Cell Physiology 56, 389-400).

<Plant>

The enhancer of the present invention can be introduced into a plant. In other words, another embodiment of this invention is directed to a plant introduced with the enhancer of this invention. Since the enhancer of this invention has an effect to enhance promoter transcription activity, the expression level of a gene located downstream of a promoter can be increased by this effect of the enhancer. For example, when the gene of interest is a gene contributing to an increase in the productivity of a plant, introduction of the enhancer of this invention into the plant can achieve an increase in the productivity of the plant.

The plant of the present invention includes not only the whole of a plantlet, but also plant organs (e.g., root, stem, leaf, petal, seed, fruit, mature embryo, immature embryo, ovule, ovary, shoot apex, anther, pollen), plant tissues (e.g., epidermis, pholoem, parenchyma, xylem, bundle), segments thereof, callus, shoot primordia, seedling, multiple shoot, hairy root, cultured root, and the like.

The plant of the present invention is a monocotyledonous plant or a dicotyledonous plant. Examples of monocotyledonous plants include, but are not limited to, Oryza sativa, Zea mays, Triticum aestivum, Hordeum vulgare, Sorghum bicolor, Saccharum officinarum, and Allium cepa. Among them, O. sativa, Z. mays, T. aestivum, and S. bicolor are preferred, with O. sativa and Z. mays being more preferred. Examples of dicotyledonous plants include, but are not limited to, Arabidopsis thaliana, Glycine max, Gossypium arboretum, Brassica campestris, Beta vulgaris, Nicotiana tabacum, Solanum lycopersicum, Raphanus sativus, Vitis vinifera, and Populus trichocarpa. Among them, A. thaliana, G. max, G. arboretum, B. campestris, N. tabacum, and S. lycopersicum are preferred, with A. thaliana, G. max, G. arboretum, and B. campestris being more preferred.

The plant of the present invention includes a plantlet obtained by growing a plant cell transfected with the enhancer of this invention, and a plant which is the progeny, offspring or clone of said plantlet, and a plant which is the progeny, offspring or clone of said plantlet, and their reproductive materials (e.g., seed, fruit, cut panicle, stem tuber, root tuber, stub, callus, protoplast). The enhancer of this invention can be transfected into a plant cell (host cell) by, for example, without particular limitation, such a procedure as described above. In the case of using the vector of this invention, the plant cell of this invention can be said as a plant transfected with the vector of this invention. Reproduction of a plantlet from a plant cell can be done by a method known to those skilled in the art depending on the type of the plant cell. Since the aforementioned reproduction techniques have been established and widely used in the technical field of this invention, these techniques can advantageously be used in this invention.

A method for regenerating a plant cell to reproduce a plantlet varies with the type of the plant cell. For example, the method described in Fujimura, et al. (Plant Tissue Culture Lett. 2: 74 (1995)) is used for Oryza sativa , and the methods described in Shillito, et al. (Bio/Technology 7: 581 (1989)) and Gorden-Kamm, et al. (Plant Cell 2: 603 (1990)) are used for Zea mays. The presence of a foreign nucleic acid transferred to a plantlet which is reproduced and planted by such a method as mentioned above can be determined by a known PCR method or the Southern hybridization method, or by analysis of the nucleotide sequence of DNA in the plantlet. In such a case, DNA extraction from a plantlet can be done by following the known method described in J. Sambrook, et al. (Molecular Cloning, 2nd ed., Cold Spring Harbor Laboratory Press, 1989; and Molecular Cloning, 3rd ed., Cold Spring Harbor Laboratory Press, 2001).

Once a plantlet transfected with the enhancer of the present invention is obtained, an offspring can be produced from the plantlet by sexual or asexual reproduction. Also, a reproductive material is obtained from the plantlet or the offspring or clone thereof, and the plantlet can be mass produced based on said reproductive material. The present invention covers a plant cell transfected with the enhancer of this invention, a plantlet comprising said cell, an offspring and clone of said plantlet, and a reproductive material from said plantlet and its offspring and clone. In other words, this invention covers “T0 generation” plants which are regenerated primary transgenic plants, and their progeny plants such as “T1 generation” plants which are seeds from T0 generation plants, as well as hybrid plants created by crossing those different generation plants, each of which is used as one parent, and progeny plants from said hybrid plants.

<Method for Enhancing Promoter Transcription Activity>

Another embodiment of the present invention is directed to a method for enhancing the transcription activity of a promoter. To be specific, the method of this invention is a method for enhancing the transcription activity of a promoter, the method comprising a step of operably inserting the enhancer of this invention as described above into the promoter, or operably linking said enhancer to the promoter.

The promoter used in the method of the present invention is not particularly limited, as described above, and is preferably a heterogeneous promoter from the enhancer of this invention. In other words, since the enhancer of this invention is derived from the Zea mays ubiquitin 1 gene promoter, the promoter used in the method of this invention is preferably a heterogeneous promoter from the Zea mays ubiquitin 1 gene promoter. In addition, the terms, materials, techniques and other matters that should be considered in relation to the inventive method are understood in accordance with the descriptions and definitions given hereinabove.

The method of the present invention is a method for enhancing the transcription activity of a promoter, and enhanced transcription activity of the promoter can be examined on the basis of the presence or absence of the enhancer inserted or linked to the promoter. In other words, when the expression level of a gene located downstream of a promoter while the enhancer is inserted into (or linked to) the promoter is larger than that while (under the same conditions except that) the enhancer is not inserted into (or linked to) the promoter, it can be determined that the transcription activity of the promoter is enhanced.

<Method for Increasing Gene Expression>

Another embodiment of the present invention is directed to a method for a method for increasing gene expression. To be specific, the method of this invention is a method for increasing gene expression, the method comprising the steps of: operably inserting the enhancer of this invention as described above into the promoter, or operably linking said enhancer to the promoter; and causing expression of a gene located downstream of the promoter. In the method of this invention, a nucleic acid molecule is incorporated in a cell, wherein the nucleic acid molecule comprises the enhancer of this invention as described above, and a promoter into which said enhancer is operably inserted or to which said enhancer is operably linked, and has a gene located downstream of the promoter. Then, the expression of a gene of interest is increased in the cell incorporating the nucleic acid molecule. The method of this invention is achieved with the use of the effect of the enhancer of this invention to enhance promoter transcription activity.

The nucleic acid molecule used in the method of the present invention comprises the enhancer of this invention, and a promoter into which said enhancer is operably inserted or to which said enhancer is operably linked. The enhancer and promoter used in the method of this invention are as described above. Also, insertion of the enhancer into the promoter, and linking of the enhancer to the promoter, are as described above. In addition, the terms, materials, techniques and other matters that should be considered in relation to the inventive method are understood in accordance with the descriptions and definitions given hereinabove.

In the method of the present invention, it is only necessary that the gene of interest should be located downstream of the promoter. The gene may be directly (adjacently) linked to the promoter, or linked via one or two or more nucleotides to the promoter. The type of the gene of interest, whose expression is to be increased, is not particularly limited—for example, a gene contributing to an increase in the productivity of a plant can be used.

In the method of the present invention, the procedure for incorporating a nucleic acid molecule in a cell is not particularly limited, and can be carried out by using the procedure for transferring the enhancer of this invention to a host cell, as described above. In other words, the nucleic acid molecule may be incorporated in a cell, as described above, by using the vector of this invention and transferring said vector to the cell. Alternatively, the nucleic acid molecule may be incorporated in a cell by transferring the enhancer of this invention or a nucleic acid comprising said enhancer to the cell, and operably inserting said enhancer into the promoter having a gene of interest located downstream thereof or operably linking said enhancer to said promoter, with the use of a gene targeting or genome editing technique. In the case where the enhancer of this invention or a nucleic acid comprising said enhancer is transferred to a cell without the use of the vector of this invention (i.e., in the latter case), the promoter into which the enhancer of this invention is operably inserted, or to which said enhancer is operably linked, is preferably an endogenous promoter present in the cell. The type of the cell to which a nucleic acid molecule is transferred is not particularly limited. The cell is preferably a plant cell, and preferred examples of plant cells are as described above.

The method of the present invention is a method for increasing gene expression, and increased gene expression can be examined on the basis of the presence or absence of the enhancer inserted or linked to the promoter. In other words, when the expression level of a gene located downstream of a promoter while the enhancer is inserted into (or linked to) the promoter is larger than that while (under the same conditions except that) the enhancer is not inserted into (or linked to) the promoter, it can be determined that the expression of the gene is increased.

<Method for Creating a Highly Productive Plant>

Another embodiment of the present invention is directed to a method for creating a highly productive plant. To be specific, the method of this invention is a method for creating a highly productive plant, the method comprising the steps of: incorporating a nucleic acid molecule in a plant cell, wherein the nucleic acid molecule comprises the enhancer of this invention as described above, and a promoter into which said enhancer is operably inserted or to which said enhancer is operably linked, and has a gene located downstream of the promoter; and creating a plant from the plant cell incorporating the nucleic acid molecule. The method of this invention is achieved with the use of the effect of the enhancer of this invention to enhance promoter transcription activity.

The nucleic acid molecule used in the method of the present invention comprises the enhancer of this invention, and a promoter into which said enhancer is operably inserted or to which said enhancer is operably linked. The enhancer and promoter contained in the nucleic acid molecule of this invention are as described above. Also, insertion of the enhancer into the promoter, and linking of the enhancer to the promoter, are as described above. In addition, the terms, materials, techniques and other matters that should be considered in relation to the inventive method are understood in accordance with the descriptions and definitions given hereinabove.

It is only necessary that the gene of interest contained in the nucleic acid molecule should be located downstream of the promoter. The gene may be directly (adjacently) linked to the promoter, or linked via one or two or more nucleotides to the promoter. The type of the gene used in the method of this invention is not particularly limited—a gene contributing to an increase in the productivity of a plant is preferably used.

In the method of the present invention, the step of incorporating a nucleic acid molecule in a plant cell is not particularly limited, and can be carried out by using the procedure for transferring the enhancer of this invention to a host cell, as described above. In other words, the nucleic acid molecule may be incorporated in a plant cell, as described above, by using the vector of this invention and transferring said vector to the plant cell. Alternatively, the nucleic acid molecule may be incorporated in a plant cell by transferring the enhancer of this invention or a nucleic acid comprising said enhancer to the plant cell, and operably inserting said enhancer into the promoter having a gene of interest located downstream thereof or operably linking said enhancer to said promoter, with the use of a gene targeting or genome editing technique. In the case where the enhancer of this invention or a nucleic acid comprising said enhancer is transferred to a plant cell without the use of the vector of this invention (i.e., in the latter case), the promoter into which the enhancer of this invention is operably inserted, or to which said enhancer is operably linked, is preferably an endogenous promoter present in the plant cell. Preferred examples of the plant cell into which a nucleic acid molecule is inserted are as described above, and are not particularly limited.

Creation of a plantlet from a plant cell can be carried out using a method known to those skilled in the art. For example, a plantlet can be created by culturing a plant cell transfected with a nucleic acid molecule to prepare a callus (cell clusters), regenerating the callus, and optionally using plant hormones (auxins and cytokines) as appropriate. Creation of a plantlet from a plant cell can also be carried out using the method described above in relation to the plant of the present invention.

The method of the present invention is a method for creating a highly productive plant, and the productivity of a created plant can be examined on the basis of the presence or absence of the enhancer inserted or linked to the promoter whose activity is to be enhanced. In other words, when the productivity of a plant while the enhancer is inserted into (or linked to) the promoter is higher than that while (under the same conditions except that) the enhancer is not inserted into (or linked to) the promoter, it can be determined that a highly productive plant is created.

### EXAMPLES

Hereunder, the present invention will be specifically described by way of working examples, but these examples are not intended to limit the technical scope of this invention. Those skilled in the art can easily make any alterations or modifications to this invention on the basis of the descriptions contained herein, and such alterations and modifications are also included in the technical scope of this invention.

### Experimental Example 1: Search for Enhancer Regions in the Zea mays Ubiquitin 1 Gene Promoter

It was presumed that potent enhancer regions might be present in a promoter of Zea mays ubiquitin 1 gene (gene ID: Zm00001d015327 (GRMZM2G409726); hereinafter referred to as “ZmUbi”). Based on this presumption, such potent enhancer regions were searched for. The method for the search is described below.

Methodology

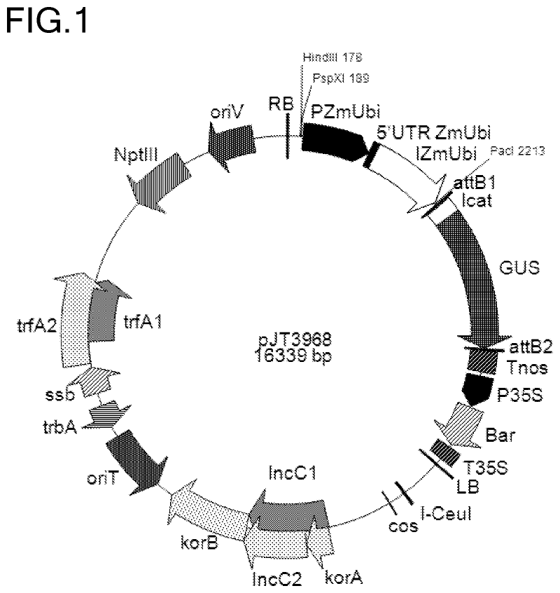

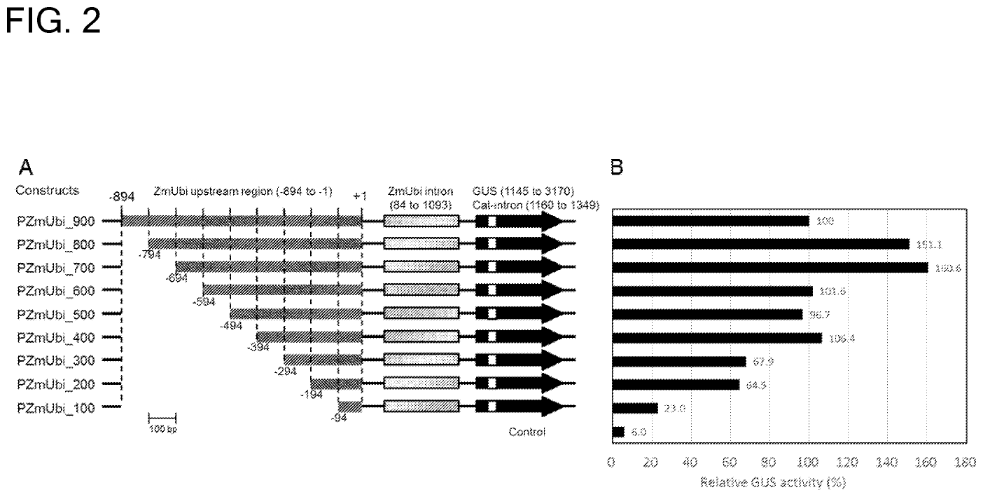

The pJT3968 vector (16339 bps; FIG. 1) tested was based on the binary vector pLC41 (Accession number: LC215698) as a backbone, and had a structure in which the gene expression cassette (PZmUbi-ZmUbiUTR-attB1-IcatGUS-attB2-Tnos) consisting of a linkage of the ZmUbi promoter, ZmUbi 5′ UTR (including intron 1), GUS gene (including Ricinus communis catalase gene intron) and the NOS terminator, and the selection marker Bar gene controlled by the cauliflower mosaic virus 35S promoter (P35S-Bar-T35S), were inserted into the multiple cloning site of the T-DNA region. Eight pJT3968 derivatives were constructed by eliminating from 100 bps up to 800 bps upstream of the ZmUbi promoter in the pJT3968 vector by an increment of 100 bps (FIG. 2A).

To be specific, eight pJT3968 derivatives were constructed by the following procedure. With the use of each of the PCR1 forward primers specific to eight pJT3968 derivatives, paired with the reverse primer pJT3968_1338R common to eight pJT3968 derivatives, as shown in the table given below, fragment 1 was amplified by PCR using the pJT3968 vector as a template. Next, for each of eight pJT3968 derivatives, with fragment 1 being used as a template, fragment 2 was amplified by PCR using a primer pair of pJT3968_174F and pJT3968_1338R. Each of the amplified fragments 2 and the pJT3968 vector were digested with the restriction enzymes HindIII and Nhel, and then ligated using the DNA ligation kit <Mighty Mix>(Takara), whereby eight pJT3968 derivatives were obtained.

TABLE 1
PCR primers used to construct pJT3968 derivatives

| Use | Primer | | Sequence | Construct |
|---|---|---|---|---|
| PCR1 | pJT3968_314Fp19 | SEQ ID NO: 46 | gtctagaggatccccaac [symbol] TGCA | PZmUbi_800 |
| | | | GTTTATCTATCTTTATACATATA | |
| PCR1 | pJT3968_414Fp19 | SEQ ID NO: 47 | gtctagaggatccccaaacATGAA | PZmUbi_700 |
| | | | CAGTTAGACATGGTCTAAAGG | |
| PCR1 | pJT3968_514Fp19 | SEQ ID NO: 48 | gtctagaggatccccaaacAAATA | PZmUbi_600 |
| | | | GCTTCACCTATATAATACTTCATC | |
| | | | C | |
| PCR1 | pJT3968_614Fp19 | SEQ ID NO: 49 | gtctagaggatccccaaacTTTTA | PZmUbi_500 |
| | | | TTCTATTTTAGCCTCTAAATTAAG | |
| PCR1 | pJT3968_714Fp19 | SEQ ID NO: 50 | gtctagaggatccccaaacAAAAT | PZmUbi_400 |
| | | | TAAACAAATACCCTTTAAGAAATT | |
| | | | AAAAAAAC | |
| PCR1 | pJT3968_814Fp19 | SEQ ID NO: 51 | gtctagaggatccccaaacTAACG | PZmUbi_300 |
| | | | GACACCAACCAGCG | |
| PCR1 | pJT3968_914Fp19 | SEQ ID NO: 52 | gtctagaggatccccaaacCGCTC | PZmUbi_200 |
| | | | CACCGTTGG | |
| PCR1 | pJT3968_1014Fp19 | SEQ ID NO: 53 | gtctagaggatccccaaacGGCAC | PZmUbi_100 |
| | | | CG [symbol] CAGCTAC | |
| PCR1, | pJT3968_1 [symbol] R | SEQ ID NO: 54 | GTGTACGAACGCTAGCAGC | All |
| PCR2 | | | | |
| PCR2 | pJT3968_174F | SEQ ID NO: 55 | cagcaagcttgcatgcctcgagtc | All |
| | | | tagaggatccccaaac | |

[symbol]

indicates data missing or illegible when filed

Next, each of the constructed eight vectors for test plots and the pJT3968 vector (construct PZmUbi_900) for a control plot was transferred to the Agrobacterium strain LBA4404 to transform 15 immature embryos of Zea mays (inbred line: A188) for each construct according to the method described in Ishida, et al., (2007, Nature Protocols 2, 1614). As a control, Agrobacterium LBA4404 containing no construct was used. Immature embryos on day 3 after inoculation were tested by MUG assay to determine GUS activity. The procedure for MUG assay is described below.

<MUG Assay>

Composition profiles of reagents
1. Extraction buffer
   50 mM phosphate buffer (pH 7.0)
   10 mM DTT
   1 mM EDTA (pH 8.0)
   0.1% sodium lauroyl sarcosinate
   0.1% Triton X-100
2. Stop buffer: 0. 2 M Na₂CO₃
3. 4-MUG stock (substrate)
   1× working solution (1 mM): 3.5 mg 4-methylumbelliferyl-β-D-glucuronide hydrate (Sigma-Aldrich)/10 mL extraction buffer
4. 4-MU calibration stock (fluorescent standard)
   1× working solution (1 mM): 2.0 mg 4-methylumbelliferone (Sigma-Aldrich)/10 mL MQ

Procedure for MUG assay
1. Fifteen immature embryos on day 3 after inoculation were ground in 300 μL of the extraction buffer, and 250 μL of the liquid extract was cryopreserved.
2. 175 μL of the extraction buffer and 275 μL of 1 mM 4-MUG were added to 100 μL of the liquid extract obtained at step 1 (in the case of pJT3968 (PZmUbi_900), a mixture of 10 μL of the liquid extract obtained at step 1+90 μL of the extraction buffer), and the mixture was reacted at 37° C.
3. After the reaction for 60 minutes, 100 μL of the reaction solution was taken out and added to 400 μL of the stop buffer. Additionally, 100 μL of the solution before the start of the reaction (after 0 min of the reaction) was also taken out and added to 400 μL of the stop buffer.
4. 200 μL of the solution obtained at step 3 was added to a 96-well plate to determine GUS activity (counts/min./mg protein) in the plate reader Tristar LB941 vTi (Berthold Technologies) (Excitation filter F370, Emission filter F450).

Results

As a result of the MUG assay, it was found, as shown in FIG. 2B, that the constructs PZmUbi_800, PZmUbi_700, PZmUbi_600, PZmUbi_500, and PZmUbi_400, which did not contain the region between −894 bp and −395 bp, showed a high level of GUS activity more than or nearly 100% relative to the GUS activity of the ZmUbi promoter full-length construct PZmUbi_900, which was taken as 100%. In contrast, it was confirmed that PZmUbi_300 not containing a −894 bp to −295 bp ZmUbi promoter region, PZmUbi_200 not containing a −894 bp to −195 bp region, and PZmUbi_100 not containing a −894 bp to −95 bp region, showed a decrease in GUS activity down to 67.9%, 64.5%, and 23.0%, respectively. These results suggested that a factor of enhancing promoter activity, i.e., enhancer factor, was present in the −394 bp to −95 bp region, especially the 100-bp region ranging from −194 bp to −95 bp, in the ZmUbi promoter.

### Experimental Example 2: Confirmation of the Effect of the Enhancers and Cis-Elements to Increase the Gene Expression Driven by the CaMV 35S Minimal Promoter

For the purpose of objectively evaluating the effects of an enhancer and cis-elements on the transcription activity of a promoter, it is a commonplace technique in the art to transfer a combination of a CaMV 35S minimal promoter and a reporter gene which visualizes gene expression to plant cells. In this experimental example, the effects of the putative enhancer region in the ZmUbi promoter, which was predicted from the results in Experimental Example 1, and a known cis-element were evaluated using GUS gene as a reporter gene, and immature Zea mays embryos as a plant material.

(1) Construction of Test Vectors

A CaMV 35S minimal promoter was chosen by reference to the reports of Ow, et al. (1987, Proceedings of the National Academy of Sciences of the United States of America 84, 4870-4874), Benfey, et al. (1990, Embo. J. 9, 1677-1684), and Ishige, et al. (1999, The Plant Journal 18, 443-448). In this experimental example, the region from −89 to +6 (transcription start site: +1) in the CaMV 35S promoter was used as a CaMV 35S minimal promoter.

The CaMV 35S minimal promoter (hereinafter referred to as “P35S-mini”) in the pBI221 vector (GenBank: AF502128) was amplified by PCR using a primer pair of P35S−89F and P35S+6R as shown in the table given below, and the amplified product was used as an insert fragment. Next, the pJT3968 vector was digested with PspXI and PacI to eliminate the ZmUbi promoter (including ZmUbi 5′ UTR and intron region), and the digested vector was used as a vector fragment. The insert fragment and vector fragment obtained above were mixed together to construct the pJT4509 vector using the In-Fusion HD Cloning Kit (Takara) (the ZmUbi promoter (including ZmUbi 5′ UTR and intron region) in the pJT3968 vector was replaced with P35S-mini). Likewise, a positive control pJT4508 vector was also constructed by inserting the CaMV 35S promoter region full-length sequence of 835 bps (including the enhancer region), P35S-FL, amplified with a primer pair of P35S−835F and P35S+6R as shown in the table given below, into the PspXI/PacI site in the pJT3968 vector.

TABLE 2-1
PCR primers used to construct vectors

| Primer | | Sequence |
|---|---|---|
| P35S − | SEQ ID | tgcctcgagtcccgggATCTCCACTGACGTAAG |
| 89F | NO: 56 | GGATG |
| P35S + | SEQ ID | acttgtgataacttaattaaCGTGTcCTCTCCA |
| 6R | NO: 57 | AATGAAATGAAC |
| P35S − | SEQ ID | tgcctcgagtcccgggAGATTAGCCTTTTCAAT |
| 835F | NO: 58 | TTCAG |

(2) Preparation of Test Constructs

Different test constructs were prepared by cloning the putative enhancer region in the ZmUbi promoter, which was predicted from the results in Experimental Example 1, or the known cis-element G-box, into the HindIII/XmaI site upstream of P35S-mini in the pJT4509 vector constructed above in (1). The procedures for preparing the constructs are described below.

(2-1) Constructs Prepared with the Enhancer Region in the ZmUbi Promoter

The fragments eZmUbi350, eZmUbi300, and eZmUbi100 were amplified by PCR. Also, the fragment eZmUbi100 (i.e., the −194 to −95 bp ZmUbi promoter region shown as a particularly potent enhancer region in Experimental Example 1) was further divided to synthesize four different 40-bp fragments (eZmUbi40a, eZmUbi40b, eZmUbi40c, and eZmUbi40d). In addition, the fragment eZmUbi40d (i.e., the −134 to −95 bp region in the ZmUbi promoter) was furthermore divided to synthesize three different 20-bp fragments (eZmUbi20d, eZmUbi20e, and eZmUbi20f). During that process, adapter sequences for cloning into the HindIII/XmaI site in the pJT4509 vector were added to the ends of the insert fragments. Then, the fragments with adapter sequences were ligated to the pJT4509 vector digested with HindIII and Xmal, to thereby prepare test constructs. The ligation was conducted using the DNA ligation kit <Mighty Mix>(Takara).

(2-2) Constructs Prepared with the Cis-Element G-Box

The core sequence (CACGTG) of G-box was reported by Ishige, et al. (1999, Plant Journal 18, 443-448). Two 10-nucleotide cis-element tetramers containing this core sequence were synthesized (G-box3(4×) and G-box10(4×)). G-box3 consists of one monomer with the nucleotide sequence of ggCACGTGcc, and G-box10 consists of one monomer with the nucleotide sequence of gcCACGTGcc. Both of them are conserved in the genome of Zea mays. The synthesized fragments G-box3(4×) and G-box10(4×) (ligated with adapter sequences for In-Fusion cloning at both ends) were inserted into the HindIII/XmaI site in the pJT4509 vector using the In-Fusion HD cloning kit (Takara), to thereby prepare test constructs.

TABLE 2-2
Enhancer regions and cis-elements inserted into P35S-mini

| Insert fragment | | Size | Sequence |
|---|---|---|---|
| eZmUbi350 | SEQ ID NO: 1 | 350 bps | AAAATTAAACAAATACCCTTTAAGAAATTAAAAAAACTA |
| | | | AGGAAACATTTTTCTTGTTTCGAGTAGATAATGCCAGCC |
| | | | TGTTAAACGCCGTCGACGAGTCTAACGGACACCAACCAG |
| | | | CGAACCAGCAGCGTCGCGTCGGGCCAAGCGAAGCAGACG |
| | | | GCACGGCATCTCTGTCGCTGCCTCTGGACCCCTCTCGAG |
| | | | AGTTCCGCTCCACCGTTGGACTTGCTCCGCTGTCGGCAT |
| | | | CCAGAAATTGCGTGGCGGAGCGGCAGACGTGAGCCGGCA |
| | | | CGGCAGGCGGCCTCCTCCTCCTCTCACGGCACCGGCAGC |
| | | | TACGGGGGGATTCCTTTCCCACCGCTCCTTCGCTTTCCC |
| eZmUbi300 | SEQ ID NO: 15 | 300 bps | AAAATTAAACAAATACCCTTTAAGAAATTAAAAAAACTA |
| | | | AGGAAACATTTTTCTTGTTTCGAGTAGATAATGCCAGCC |
| | | | TGTTAAACGCCGTCGACGAGTCTAACGGACACCAACCAG |
| | | | CGAACCAGCAGCGTCGCGTCGGGCCAAGCGAAGCAGACG |
| | | | GCACGGCATCTCTGTCGCTGCCTCTGGACCCCTCTCGAG |
| | | | AGTTCCGCTCCACCGTTGGACTTGCTCCGCTGTCGGCAT |
| | | | CCAGAAATTGCGTGGCGGAGCGGCAGACGTGAGCCGGCA |
| | | | CGGCAGGCGGCCTCCTCCTCCTCTCAC |
| eZmUbi100 | SEQ ID NO: 11 | 100 bps | CGCTCCACCGTTGGACTTGCTCCGCTGTCGGCATCCAGA |
| | | | AATTGCGTGGCGGAGCGGCAGACGTGAGCCGGCACGGCA |
| | | | GGCGGCCTCCTCCTCCTCTCAC |
| eZmUbi40a | SEQ ID NO: 2 | 40 bps | CGCTCCACCGTTGGACTTGCTCCGCTGTCGGCATCCAGA |
| | | | A |
| eZmUbi40b | SEQ ID NO: 3 | 40 bps | TCCGCTGTCGGCATCCAGAAATTGCGTGGCGGAGCGGCA |
| | | | G |
| eZmUbi40c | SEQ ID NO: 4 | 40 bps | ATTGCGTGGCGGAGCGGCAGACGTGAGCCGGCACGGCAG |
| | | | G |
| eZmUbi40d | SEQ ID NO: 5 | 40 bps | ACGTGAGCCGGCACGGCAGGCGGCCTCCTCCTCCTCTCA |
| | | | C |
| G-box3(4x) | SEQ ID NO: 59 | 40 bps | GGCACGTGCCGGCACGTGCCGGCACGTGCCGGCACGTGC |
| | | | C |
| G-box10(4x) | SEQ ID NO: 60 | 40 bps | GCCACGTGCCGCCACGTGCCGCCACGTGCCGCCACGTGC |
| | | | C |
| eZmUbi20d | SEQ ID NO: 30 | 20 bps | ACGTGAGCCGGCACGGCAGG |
| eZmUbi20e | SEQ ID NO: 31 | 20 bps | CGGCCTCCTCCTCCTCTCAC |
| eZmUbi20f | SEQ ID NO: 79 | 20 bps | GCACGGCAGGCGGCCTCCTC |

(2-3) GUS Assay of Test Constructs

Evaluation of the Enhancer Regions in the ZmUbi Promoter

Each of the different test constructs obtained above in (2-1) was transferred to the Agrobacterium strain LBA4404 to transform 10 immature Zea mays (inbred line: A188) embryos for each construct according to the method described in Ishida, et al. (2007, Nature Protocols 2, 1614). As a negative control area, a pJT4509 vector in which GUS gene was linked to P35S-mini was used for transformation. The inoculated immature embryos were subjected to GUS staining at two separate time points (1st time: day 14 after inoculation; 2nd time: day 21 after inoculation), to determine the effect to increase the gene expression driven by P35S-mini.

Evaluation of the Cis-Elements G-Boxes

Each of the two test constructs obtained above in (2-2) was transferred to the Agrobacterium strain LBA4404 to transform 7 immature embryos of Zea mays (inbred line: A188) for each construct according to the method described in Ishida, et al. (2007, Nature Protocols 2, 1614). As a negative control area, a pJT4509 vector in which GUS gene was linked to P35S-mini was used for transformation. As a positive control area, a pJT4508 vector in which GUS gene was linked to the CaMV 35S promoter region full-length sequence of 835 bps (including the CaMV 35S enhancer sequence), P35S-FL, was used for transformation. The inoculated immature embryos were subjected to GUS staining on day 4 after inoculation, to determine the effect to increase the gene expression driven by P35S-mini.

The procedure for GUS staining is described below.

<GUS Staining>

Composition Profiles of Reagents
1. 50 mM NaPi buffer (pH 6.8)
   NaH₂PO₄.H₂O: 6.66 g/L
   Na₂HPO₄.12H₂O: 17.66 g/L
2. NaPi buffer+Triton
   50 mM NaPi buffer (pH 6.8): 198 mL
   10% Triton X-100: 2 mL
3. X-Gluc solution
   5-Bromo-4-chloro-3-indolyl-β-D-glucuronide: 100 mg
   Ethylene glycol monomethyl ether: 2 mL
4. X-Gluc reaction solution
   NaPi buffer+Triton: 790 μL
   Methanol: 200 μL
   X-Gluc solution: 10 μL

Procedure for GUS Staining
1. Immature embryos or calli after inoculation were immersed in the NaPi buffer+Triton solution.
2. After the NaPi buffer+Triton solution was removed, the X-Gluc reaction solution was added, and the mixture was reacted at 37° C. overnight.
3. After the X-Gluc reaction solution was removed, distilled water was added.
4. The diluted sample was transferred to an agar medium, observed on a stereomicroscope (OLYMPUS SZX12), and photographed.

For each test area, immature embryos were counted depending on the intensity of GUS staining of individual embryos, according to the five-grade evaluation scale detailed below.
+++: A very dark blue GUS stain is observed.
++: A dark blue GUS stain is observed.
+: A blue GUS stain is observed.
±: A slight GUS stain (very light blue) is observed.
−: No GUS stain is observed.

Results

As a result of the GUS staining, it was confirmed, as shown in the tables given below, that enhanced GUS activity was observed in all of the test areas as compared to P35S-mini in the control areas. According to a previous report on the evaluation of G-box3 and G-box10 in tobacco plants, there was a difference in GUS activity level between the two G-boxes (Ishige, et al., 1999, Plant Journal 18, 443-448). However, in this experimental example using immature Z. mays embryos, no significant difference was found between the two G-boxes in terms of the effect to increase GUS expression driven by P35S-mini. Additionally, it should be noted that in the results shown in the tables below, the number of immature embryos also includes calli formed on the immature embryos.

TABLE 2-3
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| G-box3(4×) | 40 bps | 0 | 0 | 0 | 0 | 7 | 7 |
| G-box10(4×) | 40 bps | 0 | 0 | 0 | 0 | 7 | 7 |
| P35S-FL | 835 bps | 0 | 0 | 0 | 0 | 6 | 6 |
| P35S-mini | — | 0 | 2 | 5 | 0 | 0 | 7 |

TABLE 2-4
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi100 | 100 bps | 0 | 0 | 6 | 4 | 0 | 10 |
| eZmUbi300 | 300 bps | 0 | 0 | 9 | 1 | 0 | 10 |
| eZmUbi350 | 350 bps | 0 | 0 | 8 | 1 | 1 | 10 |
| P35S-FL | 835 bps | 0 | 0 | 7 | 3 | 0 | 10 |
| P35S-mini | — | 3 | 2 | 5 | 0 | 0 | 10 |

TABLE 2-5
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi100 | 100 bps | 2 | 1 | 6 | 0 | 1 | 10 |
| eZmUbi40a | 40 bps | 5 | 0 | 4 | 1 | 0 | 10 |
| eZmUbi40b | 40 bps | 6 | 1 | 3 | 0 | 0 | 10 |
| eZmUbi40c | 40 bps | 3 | 1 | 6 | 0 | 0 | 10 |
| eZmUbi40d | 40 bps | 2 | 1 | 2 | 5 | 0 | 10 |
| P35S-mini | — | 7 | 0 | 1 | 1 | 0 | 9 |

TABLE 2-6
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi20d | 20 bps | 1 | 2 | 5 | 2 | 0 | 10 |
| eZmUbi20e | 20 bps | 3 | 3 | 3 | 1 | 0 | 10 |
| eZmUbi20f | 20 bps | 4 | 3 | 3 | 0 | 0 | 10 |
| P35S-mini | — | 4 | 4 | 2 | 0 | 0 | 10 |

### Experimental Example 3: Evaluation Test of the Effect of Enhancers and Cis-Elementsto Increase the Gene Expression Driven by the ZmBIL7 Promoter

In Experimental Example 2 as mentioned above, different enhancers and cis-elements were demonstrated to have an effect to increase the gene expression driven by P35S-mini. In this experimental example, each or combinations of these enhancers and cis-elements were evaluated for the effect to increase the gene expression driven by the promoter of ZmBIL7 gene (gene ID: Zm00001d051884 (GRMZM2G143854)) in immature Zea mays embryos.

(1) Construction of a GUS expression cassette-containing vector controlled by the ZmBIL7 promoter

The ZmBIL7 promoter region isolated was the 3000-bp region from 173461088 bp to 173458089 bp in chromosome 4 of the Z. mays B73 genome sequence (Assembly: B73 RefGen_v4) publicly available on the website of EnsemblPlants (http://plants.ensembl.org/index.html).

DNA was extracted from Z. mays leaves (inbred line: B73), and the region containing the 3000-bp ZmBIL7 promoter and 714-bp 5′ UTR downstream thereof was amplified by PCR using a primer pair of XmaI_ZmBIL7pro-3714F and ZmBIL7pro-1R as shown in the table given below, and Tks Gflex DNA Polymerase (Takara). The amplified fragment was cloned between the PspXI and PacI sites in the pJT3968 vector using the In-Fusion HD cloning kit (Takara), to thereby prepare the pJT4712 vector. As a result of construction of this plasmid, the ZmUbi promoter (including ZmUbi gene 5′ UTR downstream thereof) in the pJT3968 vector was replaced with the ZmBIL7 promoter (including ZmBIL7 gene 5′ UTR downstream thereof), and also, the restriction enzyme XmaI recognition site (corresponding to the region from −3006 bp to −3001 bp) was added immediately upstream of the ZmBIL7 promoter. Further, with the pJT4712 vector being used as a template, PCR was performed using a primer pair of ZB7pIG_2588F and ZB7pUIG_3212R, to thereby amplify fragment 1 containing the 615-bp region at the 3′ end of the ZmBIL7 promoter. Also, with the pJT3968 vector being used as a template, PCR was performed using a primer pair of ZB7pUIG_3193F and ZB7pUIG_4321R, to thereby amplify fragment 2 containing 1093-bp ZmUbi 5′ UTR. Next, fragments 1 and 2 were mixed together, and subjected to overlap extension PCR using a primer pair of ZB7pIG_2588F and ZB7pUIG_4321R, to thereby prepare fragment 3 in which fragments 1 and 2 were linked together. Fragment 3 was digested with StuI and PacI, and ligated to the pJT4712 vector digested with the same restriction enzymes, to thereby obtain the pJT4713 vector in which ZmUbi gene 5′ UTR was linked downstream of the ZmBIL7 promoter.

TABLE 3-1
Primers used to construct vectors.

| Primer | | Sequence |
|---|---|---|
| XmaI_ZmBIL7pro-3714F | SEQ ID NO: 61 | cttgcatgcctcgagtcccgggTGATGCCAAACATGTCC |
| | | CTCAC |
| ZmBIL7pro-1R | SEQ ID NO: 62 | acttgtgataacttaattaaGATTGCGGCAGCAAAGAGC |
| | | AAGC |
| ZB7pIG_2588F | SEQ ID NO: 63 | GGGCTGGGCGCTG |
| ZB7pUIG_3212R | SEQ ID NO: 64 | GGTTGGGGAAaaaggaagggttgcaggcttg |
| ZB7pUIG_3193F | SEQ ID NO: 65 | cccttcctttTTCCCCAACCTCGTGTTGTTC |
| ZB7pUIG_4321R | SEQ ID NO: 66 | GATAACTTAATTAATCTAGAGTCGACCTG |

(2) Construction of Vectors Having an enhancer and/or a cis-element inserted into the −105 bp site in the ZmBIL7 promoter

By following the procedure described later, different GUS assay vectors were constructed, in which any of the enhancer regions whose effect was demonstrated in Experimental Example 2, or any combination of any of the enhancer regions and cis- elements, was inserted into the ZmBIL7 promoter in the pJT4713 vector. The nucleotide sequences inserted into said ZmBIL7 promoter are shown in the tables given below.

TABLE 3-2A
Enhancers and/or cis-elements inserted into ZmBIL7 promoter

| Insert fragment | | Size | Sequence |
|---|---|---|---|
| eZmUbi100 | SEQ ID NO: 11 | 100 bps | CGCTCCACCGTTGGACTTGCTCCGCTGTCGGCATCCAG |
| | | | AAATTGCGTGGCGGAGCGGCAGACGTGAGCCGGCACGG |
| | | | CAGGCGGCCTCCTCCTCCTCTCAC |
| eZmUbi300 | SEQ ID NO: 15 | 300 bps | AAAATTAAACAAATACCCTTTAAGAAATTAAAAAAACT |
| | | | AAGGAAACATTTTTCTTGTTTCGAGTAGATAATGCCAG |
| | | | CCTGTTAAACGCCGTCGACGAGTCTAACGGACACCAAC |
| | | | CAGCGAACCAGCAGCGTCGCGTCGGGCCAAGCGAAGCA |
| | | | GACGGCACGGCATCTCTGTCGCTGCCTCTGGACCCCTC |
| | | | TCGAGAGTTCCGCTCCACCGTTGGACTTGCTCCGCTGT |
| | | | CGGCATCCAGAAATTGCGTGGCGGAGCGGCAGACGTGA |
| | | | GCCGGCACGGCAGGCGGCCTCCTCCTCCTCTCAC |
| eZmUbi350 | SEQ ID NO: 1 | 350 bps | AAAATTAAACAAATACCCTTTAAGAAATTAAAAAAACT |
| | | | AAGGAAACATTTTTCTTGTTTCGAGTAGATAATGCCAG |
| | | | CCTGTTAAACGCCGTCGACGAGTCTAACGGACACCAAC |
| | | | CAGCGAACCAGCAGCGTCGCGTCGGGCCAAGCGAAGCA |
| | | | GACGGCACGGCATCTCTGTCGCTGCCTCTGGACCCCTC |
| | | | TCGAGAGTTCCGCTCCACCGTTGGACTTGCTCCGCTGT |
| | | | CGGCATCCAGAAATTGCGTGGCGGAGCGGCAGACGTGA |
| | | | GCCGGCACGGCAGGCGGCCTCCTCCTCCTCTCACGGCA |
| | | | CCGGCAGCTACGGGGGATTCCTTTCCCACCGCTCCTTC |
| | | | GCTTTCCC |
| G-box10(4x) | SEQ ID NO: 60 | 40 bps | GCCACGTGCCGCCACGTGCCGCCACGTGCCGCCACGTG |
| | | | CC |
| eZmUbi100 + | SEQ ID NO: 67 | 140 bps | CGCTCCACCGTTGGACTTGCTCCGCTGTCGGCATCCAG |
| Gbox10(4x) | | | AAATTGCGTGGCGGAGCGGCAGACGTGAGCCGGCACGG |
| | | | CAGGCGGCCTCCTCCTCCTCTCACgcCACGTGccgcCA |
| | | | CGTGccgcCACGTGccgcCACGTGcc |
| Gbox10(4x) + | SEQ ID NO: 68 | 140 bps | gcCACGTGccgcCACGTGccgcCACGTGccgcCACGTG |
| eZmUbi100 | | | ccCGCTCCACCGTTGGACTTGCTCCGCTGTCGGCATCC |
| | | | AGAAATTGCGTGGCGGAGCGGCAGACGTGAGCCGGCAC |
| | | | GGCAGGCGGCCTCCTCCTCCTCTCAC |

TABLE 3-2B

| | | | |
|---|---|---|---|
| eZmUbi300 + | SEQ ID NO: 69 | 340 bps | AAAATTAAACAAATACCCTTTAAGAAATTAAAAAAACT |
| Gbox10(4x) | | | AAGGAAACATTTTTCTTGTTTCGAGTAGATAATGCCAG |
| | | | CCTGTTAAACGCCGTCGACGAGTCTAACGGACACCAAC |
| | | | CAGCGAACCAGCAGCGTCGCGTCGGGCCAAGCGAAGCA |
| | | | GACGGCACGGCATCTCTGTCGCTGCCTCTGGACCCCTC |
| | | | TCGAGAGTTCCGCTCCACCGTTGGACTTGCTCCGCTGT |
| | | | CGGCATCCAGAAATTGCGTGGCGGAGCGGCAGACGTGA |
| | | | GCCGGCACGGCAGGCGGCCTCCTCCTCCTCTCACgcCA |
| | | | CGTGccgcCACGTGccgcCACGTGccgcCACGTGcc |
| eZmUbi300 + | SEQ ID NO: 70 | 380 bps | AAAATTAAACAAATACCCTTTAAGAAATTAAAAAAACT |
| Gbox10(8x) | | | AAGGAAACATTTTTCTTGTTTCGAGTAGATAATGCCAG |
| | | | CCTGTTAAACGCCGTCGACGAGTCTAACGGACACCAAC |
| | | | CAGCGAACCAGCAGCGTCGCGTCGGGCCAAGCGAAGCA |
| | | | GACGGCACGGCATCTCTGTCGCTGCCTCTGGACCCCTC |
| | | | TCGAGAGTTCCGCTCCACCGTTGGACTTGCTCCGCTGT |
| | | | CGGCATCCAGAAATTGCGTGGCGGAGCGGCAGACGTGA |
| | | | GCCGGCACGGCAGGCGGCCTCCTCCTCCTCTCACgcCA |
| | | | CGTGccgcCACGTGccgcCACGTGccgcCACGTGccgc |
| | | | CACGTGccgcCACGTGccgcCACGTGccgcCACGTGcc |
| eZmUbi350 + | SEQ ID NO: 71 | 390 bps | AAAATTAAACAAATACCCTTTAAGAAATTAAAAAAACT |
| Gbox10(4x) | | | AAGGAAACATTTTTCTTGTTTCGAGTAGATAATGCCAG |
| | | | CCTGTTAAACGCCGTCGACGAGTCTAACGGACACCAAC |
| | | | CAGCGAACCAGCAGCGTCGCGTCGGGCCAAGCGAAGCA |
| | | | GACGGCACGGCATCTCTGTCGCTGCCTCTGGACCCCTC |
| | | | TCGAGAGTTCCGCTCCACCGTTGGACTTGCTCCGCTGT |
| | | | CGGCATCCAGAAATTGCGTGGCGGAGCGGCAGACGTGA |
| | | | GCCGGCACGGCAGGCGGCCTCCTCCTCCTCTCACGGCA |
| | | | CCGGCAGCTACGGGGGATTCCTTTCCCACCGCTCCTTC |
| | | | GCTTTCCCgcCACGTGccgcCACGTGccgcCACGTGcc |
| | | | gcCACGTGcc |

TABLE 3-2C

| Insert fragment | | Size | Sequence |
|---|---|---|---|
| eZmUbi100 + | SEQ ID NO: 80 | 130 bps | CGCTCCACCGTTGGACTTGCTCCGCTGTCGGCATCCAG |
| Gbox10(3x) | | | AAATTGCGTGGCGGAGCGGCAGACGTGAGCCGGCACGG |
| | | | CAGGCGGCCTCCTCCTCCTCTCACgcCACGTGccgcCA |
| | | | CGTGccgcCACGTGcc |
| eZmUbi100 + | SEQ ID NO: 81 | 120 bps | CGCTCCACCGTTGGACTTGCTCCGCTGTCGGCATCCAG |
| Gbox10(2x) | | | AAATTGCGTGGCGGAGCGGCAGACGTGAGCCGGCACGG |
| | | | CAGGCGGCCTCCTCCTCCTCTCACgcCACGTGccgcCA |
| | | | CGTGcc |
| eZmUbi100 + | SEQ ID NO: 82 | 110 bps | CGCTCCACCGTTGGACTTGCTCCGCTGTCGGCATCCAG |
| Gbox10(1x) | | | AAATTGCGTGGCGGAGCGGCAGACGTGAGCCGGCACGG |
| | | | CAGGCGGCCTCCTCCTCCTCTCACgcCACGTGcc |
| eZmUbi40d | SEQ ID NO: 5 | 40 bps | ACGTGAGCCGGCACGGCAGGCGGCCTCCTCCTCCTCTC |
| | | | AC |
| eZmUbi20d | SEQ ID NO: 30 | 20 bps | ACGTGAGCCGGCACGGCAGG |
| eZmUbi20e | SEQ ID NO: 31 | 20 bps | CGGCCTCCTCCTCCTCTCAC |
| eZmUbi20f | SEQ ID NO: 79 | 20 bps | GCACGGCAGGCGGCCTCCTC |
| eZmUbi20d + | SEQ ID NO: 83 | 60 bps | ACGTGAGCCGGCACGGCAGGgcCACGTGccgcCACGTG |
| Gbox10(4x) | | | ccgcCACGTGccgcCACGTGcc |
| eZmUbi20e + | SEQ ID NO: 84 | 60 bps | CGGCCTCCTCCTCCTCTCACgcCACGTGccgcCACGTG |
| Gbox10(4x) | | | ccgcCACGTGccgcCACGTGcc |
| eZmUbi20f + | SEQ ID NO: 85 | 60 bps | GCACGGCAGGCGGCCTCCTCgcCACGTGccgcCACGTG |
| Gbox10(4x) | | | ccgcCACGTGccgcCACGTGcc |

It is generally considered that the location of a cis-element on a promoter is preferably at a site about 50 bp upstream from the core promoter region or TATA-box, which are located about 50 bp upstream of the transcription start site (Aysha, et al., 2018, Mol. Biotechnol. 60, 608-620; and Pandiarajan and Grover, 2018, Plant Science 277, 132-138). Thus, in the present invention, the −105 bp site in the ZmBIL7 promoter was selected as an insertion site. In order to make an insertion site, the pJT4713 vector was modified as a first step. The PCR primers used for construction are shown in the table given below. With the pJT4713 vector being used as a template, PCRs were performed using a primer pair of ZBp3000u_2584F and GBoxcore1_4_R, or a primer pair of ZBp3000u_4309R and GBoxcore1_4_F, to thereby prepare fragments 1 and 2, respectively. Next, fragments 1 and 2 were mixed together, and subjected to over-extension PCR using a primer pair of ZBp3000u_2584F and ZBp3000u_4309R, to thereby prepare fragment 3 in which fragments 1 and 2 were linked together. Fragment 3 was inserted between the PstI and Pstl sites in the pJT4713 vector using the In-Fusion HD cloning kit (Takara), to construct the pJT4714 vector. As a result of construction of this vector, the −108 to −103 bp nucleotide sequence (CAAGTC) in the ZmBIL7 promoter of the pJT4713 vector was replaced with the PmlI recognition site (CACGTG) in the pJT4714 vector. Next, the adapter sequences were added to each of the insert fragments shown in the tables given above by PCR. Then, the fragments were inserted into the PmlI site in the pJT4714 vector using the In-Fusion HD cloning kit (Takara), to thereby prepare test constructs.

TABLE 3-3
PCR primers used to construct pJT4714 vector

| Primer | | Sequence |
|---|---|---|
| ZBp3000u_2584F | SEQ ID | GCTAGGGCTGGGCGCTGC |
| | NO: 72 | |
| GBoxcore1_4_R | SEQ ID | ccgtgcctaccacgtgtg |
| | NO: 73 | aggttataagag |
| GBoxcore1_4_F | SEQ ID | ctcttataacctcacacg |
| | NO: 74 | tggtaggcacgg |
| ZBp3000u_4309R | SEQ ID | aatctagagtcgacctgc |
| | NO: 75 | agaagtaacacc |

(3) GUS Assay of Test Constructs

Each of the different test constructs obtained above was transferred to the Agrobacterium strain LBA4404 to transform 10 immature embryos of Z. mays (inbred line: A188) for each construct according to the method described in Ishida, et al. (2007, Nature Protocols 2, 1614). As a negative control area, a pJT4713 vector in which GUS gene was linked to the ZmBIL7 promoter was used for transformation. After inoculation, the immature embryos were subjected to GUS staining at two separate time points (1st time: day 13 to day 17 after inoculation; 2nd time: day 20 to day 22 after inoculation), to determine the effect to increase the gene expression driven by the ZmBIL7 promoter. Additionally, GUS staining was performed by using the same procedure as in Experimental Example 2, and evaluation of the intensity of GUS staining was also made according to the same evaluation scale as in Experimental Example 2.

Results

As a result of the GUS staining, it was demonstrated, as shown in the tables given below, that in the cases where each of the tested enhancer regions was inserted alone into PZmBIL7, significantly enhanced GUS activity was observed in some test areas while GUS activity was not so significantly enhanced in other test areas. However, it was found that in the cases where some enhancer regions which did not significantly enhance GUS activity when used alone were combined with a G-box cis-element and inserted into PZmBIL7, GUS activity was significantly enhanced. In the cases where other enhancer regions which significantly enhanced GUS activity when used alone were combined with a G-box cis-element, GUS activity was still more significantly enhanced than in the cases where those enhancer regions were inserted alone. In particular, significant enhancement of GUS activity was observed markedly in the cases where a cis-element was linked downstream of the enhancer regions. Additionally, it should be noted that in the results shown in the tables below, the number of immature embryos also includes calli formed on the immature embryos.

TABLE 3-4
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi100 | 100 bps | 2 | 8 | 0 | 0 | 0 | 10 |
| eZmUbi100 + | 140 bps | 0 | 0 | 4 | 6 | 0 | 10 |
| Gbox10(4×) | | | | | | | |
| Gbox10(4×) + | 140 bps | 0 | 2 | 8 | 0 | 0 | 10 |
| eZmUbi100 | | | | | | | |
| PZmUbi_900 | — | 0 | 0 | 0 | 2 | 8 | 10 |
| PZmBIL7 | — | 10 | 0 | 0 | 0 | 0 | 10 |

TABLE 3-5
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| Gbox10(4×) | 40 bps | 8 | 1 | 1 | 0 | 0 | 10 |
| PZmBIL7 | — | 10 | 0 | 0 | 0 | 0 | 10 |

TABLE 3-6
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi100 + | 140 bps | 0 | 0 | 5 | 5 | 0 | 10 |
| Gbox10(4×) | | | | | | | |
| eZmUbi300 | 300 bps | 1 | 0 | 5 | 4 | 0 | 10 |
| eZmUbi300 + | 340 bps | 0 | 1 | 1 | 7 | 1 | 10 |
| Gbox10(4×) | | | | | | | |
| PZmUbi_900 | — | 0 | 0 | 0 | 3 | 7 | 10 |
| PZmBIL7 | — | 9 | 1 | 0 | 0 | 0 | 10 |

TABLE 3-7
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi350 | 350 bps | 0 | 2 | 7 | 0 | 1 | 10 |
| eZmUbi350 + | 390 bps | 0 | 1 | 5 | 1 | 2 | 9 |
| Gbox10(4×) | | | | | | | |
| eZmUbi300 + | 380 bps | 0 | 1 | 4 | 5 | 0 | 10 |
| Gbox10(8×) | | | | | | | |
| PZmUbi_900 | — | 0 | 1 | 2 | 5 | 2 | 10 |
| PZmBIL7 | — | 9 | 1 | 0 | 0 | 0 | 10 |

TABLE 3-8
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi100 + | 140 bps | 0 | 0 | 8 | 1 | 1 | 10 |
| Gbox10(4×) | | | | | | | |
| eZmUbi100 + | 130 bps | 0 | 1 | 5 | 2 | 2 | 10 |
| Gbox10(3×) | | | | | | | |
| eZmUbi100 + | 120 bps | 0 | 2 | 5 | 3 | 0 | 10 |
| Gbox10(2×) | | | | | | | |
| eZmUbi100 + | 110 bps | 1 | 0 | 8 | 1 | 0 | 10 |
| Gbox10(1×) | | | | | | | |
| PZmBIL7 | — | 9 | 1 | 0 | 0 | 0 | 10 |
| eZmUbi300 + | 340 bps | 0 | 0 | 3 | 5 | 2 | 10 |
| Gbox10(4×) | | | | | | | |

TABLE 3-9
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi40d | 40 bps | 5 | 3 | 2 | 0 | 0 | 10 |
| eZmUbi40d + | 80 bps | 1 | 1 | 7 | 1 | 0 | 10 |
| Gbox10(4×) | | | | | | | |
| eZmUbi20e | 20 bps | 7 | 2 | 1 | 0 | 0 | 10 |
| eZmUbi20e + | 60 bps | 0 | 0 | 7 | 3 | 0 | 10 |
| Gbox10(4×) | | | | | | | |
| PZmBIL7 | — | 10 | 0 | 0 | 0 | 0 | 10 |
| eZmUbi300 + | 340 bps | 0 | 0 | 1 | 7 | 2 | 10 |
| Gbox10(4×) | | | | | | | |

TABLE 3-10
GUS staining of inoculated immature embryos

| | | Number of immature embryos | | | | | |
|---|---|---|---|---|---|---|---|
| | | depending on the degree of GUS staining | | | | | |
| Construct | Size | − | ± | + | ++ | +++ | Total |
| eZmUbi20d | 20 bps | 8 | 1 | 1 | 0 | 0 | 10 |
| eZmUbi20d + | 60 bps | 0 | 0 | 6 | 3 | 1 | 10 |
| Gbox10(4×) | | | | | | | |
| eZmUbi20f | 20 bps | 9 | 1 | 0 | 0 | 0 | 10 |
| eZmUbi20f + | 60 bps | 2 | 3 | 4 | 0 | 1 | 10 |
| Gbox10(4×) | | | | | | | |
| PZmBIL7 | — | 9 | 1 | 0 | 0 | 0 | 10 |
| eZmUbi300 + | 340 | 0 | 1 | 3 | 4 | 2 | 10 |
| Gbox10(4×) | | | | | | | |

### Experimental Example 4: Biomass Evaluation in Oryza sativa Transformants

It has been reported that when Oryza sativa or Arabidopsis thaliana BIL7 gene is linked to a high-expression promoter and highly expressed in O. sativa, the biomass of O. sativa is increased (International Patent Publication No. WO 2016/056650). Thus, transformation vectors were prepared by linking a genomic fragment of ZmBIL7 gene downstream of the ZmBIL7 promoter into which each of the fragments demonstrated to have an enhancer effect in Experimental Example 3 were inserted. The prepared transformation vectors were transferred to O. sativa to evaluate the biomass of O. sativa.

The enhancer fragments used to construct transformation vectors are shown in the table given below. The pJT4627 vector used for construction was based on pLC41 (Accession number: LC215698) as a backbone, and harbored two T-DNA regions. The first T-DNA region contained a gene expression cassette (Pnos-Barnase-T35S) for the negative selection marker Barnase gene (including O. sativa Rf-1 gene intron 5) controlled by the NOS promoter. The second T-DNA region contained a gene expression cassette (PZmBIL7-ZmBIL7genome) in which a genomic fragment of ZmBIL7 gene was linked downstream of the ZmBIL7 promoter, and a gene expression cassette (P35S-Icat-HPT-T35S) linked to the selection marker HPT gene controlled by the 35S promoter. The linked genomic fragment of ZmBIL7 gene was the 4899-bp region from 173458088 bp to 173453190 bp in chromosome 4 of the Z. mays B73 genome sequence (Assembly: B73 RefGen_v4) publicly available on the website of EnsemblPlants (http://plants.ensembl.org/index.html), and contained 5′ UTR, a protein-coding region, and 3′ UTR. In addition, the pJT4631 vector in which ZmBIL7 gene 5′ UTR in the pJT4627 vector was replaced with ZmUbi gene 5′ UTR (including intron 1) was also used for construction. Test constructs were prepared by replacing the ZmBIL7 promoter, into which each of the enhancer fragments shown in the table below and constructed in Experimental Example 3 was inserted, with the ZmBIL7 promoter in each of the aforementioned two vectors. O. sativa (the variety “Yukihikari”) was transformed with each of the thus- prepared four test constructs and control construct (i.e., a pJT4627 vector with no enhancer inserted into the ZmBIL7 promoter) using the Agrobacterium strain LBA4404. The transformation of O. sativa was performed according to the method described in Hiei, et al. (2008, Plant J., 6: 271-282). Cultivation and evaluation of primary transformants (T0 generation) were performed in a dedicated greenhouse for recombinant plants in the Plant Innovation Center of Japan Tobacco Inc. The conditions employed for cultivation of O. sativa were long-day conditions with a day length of 14.5 hours and with a daytime temperature of 28° C. and a nighttime temperature of 20° C. For each of the groups of recombinant individuals transformed with the four test constructs and one control construct, 49 plantlets were transferred to a connected seedling pot tray (49 holes). On days 10 and 17 after potting, 49 plantlets in each group were measured for plant height. On day 18 after potting, 30 well-grown plantlets were selected in each group, transferred one by one to polypots (diameter: 12 cm, volume: 830 cc), and cultivated until harvest. The number of days to heading and the total rough rice grain weight after harvest were measured.

TABLE 4-1

| Insert fragment [symbol] | | Size [symbol] | Sequence [symbol] |
|---|---|---|---|
| eZmUbi100 + ↓ | SEQ ID | 140 bps [symbol] | CGCTCCACCGTTGGACTTGCTCCGCTGTCGGCATCC |
| Gbox10(4x) [symbol] | NO: 67 [symbol] | | AGAAATTGCGTGGCGGAGCGGCAGACGTGAGCCGGC |
| | | | ACGGCAGGCGGCCTCCTCCTCCTCTCACgcCACGTG |
| | | | ccgcCACGTGccgcCACGTGccgcCACGTGcc [symbol] |
| eZmUbi300 + ↓ | SEQ ID | 340 bps [symbol] | AAAATTAAACAAATACCCTTTAAGAAATTAAAAAAA |
| Gbox10(4x) [symbol] | NO: 69 [symbol] | | CTAAGGAAACATTTTTCTTGTTTCGAGTAGATAATG |
| | | | CCAGCCTGTTAAACGCCGTCGACGAGTCTAACGGAC |
| | | | ACCAACCAGCGAACCAGCAGCGTCGCGTCGGGCCAA |
| | | | GCGAAGCAGACGGCACGGCATCTCTGTCGCTGCCTC |
| | | | TGGACCCCTCTCGAGAGTTCCGCTCCACCGTTGGAC |
| | | | TTGCTCCGCTGTCGGCATCCAGAAATTGCGTGGCGG |
| | | | AGCGGCAGACGTGAGCCGGCACGGCAGGCGGCCTCC |
| | | | TCCTCCTCTCACgcCACGTGccgcCACGTGccgcCA |
| | | | CGTGccgcCACGTGcc [symbol] |

The results of this experiment are as shown in the tables given below. All of the groups of plantlets transformed with the four constructs used in this experiment had a higher plant height than the control group, on both of days 10 and 17 after potting. The number of days to heading in the four test construct groups was comparable to that in the control group. The total rough rice grain weight in the four test construct groups was greater by 0.44 to 2.23 g than that in the control group. These results suggested that utilization of the enhancers provided according to the present invention results in an increase in the biomass of plants and allows for the creation of highly productive plants.

TABLE 4-2

| | Insert | | Plant | Plant |
|---|---|---|---|---|
| | fragment | | height on | height on |
| | into ZmBIL7 | Vector | day 10 after | day 17 after |
| Construct | promoter | backbone | potting (cm) | potting (cm) |
| 1 | eZmUbi100 + | pJT4627 | 26.2 | 36.5 |
| | Gbox10(4x) | | | |
| 2 | eZmUbi100 + | pJT4631 | 26.5 | 39.1 |
| | Gbox10(4x) | | | |
| 3 | eZmUbi300 + | pJT4627 | 27.7 | 39.3 |
| | Gbox10(4x) | | | |
| 4 | eZmUbi300 + | pj T4631 | 24.6 | 36.4 |
| | Gbox10(4x) | | | |
| Control | — | pJT4627 | 23.9 | 33.6 |

TABLE 4-3

| | Insert | | | Total |
|---|---|---|---|---|
| | fragment | | Number | rough rice |
| | into ZmBIL7 | Vector | of days | grain |
| Construct | promoter | backbone | to heading | weight (g) |
| 1 | eZmUbi100 + | pJT4627 | 42.5 | 6.43 |
| | Gbox10(4x) | | | |
| 2 | eZmUbi100 + | pJT4631 | 42.5 | 7.91 |
| | Gbox10(4x) | | | |
| 3 | eZmUbi300 + | pJT4627 | 42.5 | 7.44 |
| | Gbox10(4x) | | | |
| 4 | eZmUbi300 + | pj T4631 | 43.1 | 8.22 |
| | Gbox10(4x) | | | |
| Control | — | pJT4627 | 42.9 | 5.99 |

### INDUSTRIAL APPLICABILITY

The present invention is useful in all industrial fields utilizing gene expression. In one instance, this invention is useful in industrial fields in which the biomass of plants can be utilized, such as the fields of foods, energy and environment. By utilizing this invention, the transcription activity of promoters in genes can be enhanced, and the expression levels of genes can be increased.

1. An enhancer comprising the following polynucleotide (i) or (ii):
(i) a polynucleotide comprising a sequence of at least 20 consecutive nucleotides in the region of nucleotides 201 to 300 in SEQ ID NO: 1; or
(ii) a polynucleotide that consists of a nucleotide sequence having at least 90% sequence identify to that of the polynucleotide (i), and has an effect to enhance promoter transcription activity.

2. The enhancer according to claim 1, wherein the polynucleotide (i) comprises a sequence of at least 40 consecutive nucleotides in the region of nucleotides 201 to 300 in SEQ ID NO: 1.

3. The enhancer according to claim 1, wherein the polynucleotide (i) comprises a sequence of at least 60 consecutive nucleotides in the region of nucleotides 201 to 300 in SEQ ID NO: 1.

4. The enhancer according to claim 1, wherein the polynucleotide (i) comprises a sequence of at least 80 consecutive nucleotides in the region of nucleotides 201 to 300 in SEQ ID NO: 1.

5. The enhancer according to claim 1, wherein the polynucleotide (i) comprises a nucleotide sequence represented by the region of nucleotides 261 to 280 in SEQ ID NO: 1, a nucleotide sequence represented by the region of nucleotides 271 to 290 in SEQ ID NO: 1, or a nucleotide sequence represented by the region of nucleotides 281 to 300 in SEQ ID NO: 1.

6. The enhancer according to claim 1, wherein the polynucleotide (i) comprises a nucleotide sequence represented by the region of nucleotides 201 to 240 in SEQ ID NO: 1, a nucleotide sequence represented by the region of nucleotides 221 to 260 in SEQ ID NO: 1, a nucleotide sequence represented by the region of nucleotides 241 to 280 in SEQ ID NO: 1, or a nucleotide sequence represented by the region of nucleotides 261 to 300 in SEQ ID NO: 1.

7. The enhancer according to claim 1, wherein the polynucleotide (i) comprises a nucleotide sequence represented by the region of nucleotides 201 to 300 in SEQ ID NO: 1.

8. The enhancer according to claim 1, wherein the polynucleotide (i) comprises a nucleotide sequence represented by the region of nucleotides 1 to 300 in SEQ ID NO: 1.

9. The enhancer according to claim 1, wherein the polynucleotide (i) comprises a nucleotide sequence represented by SEQ ID NO: 1.

10. The enhancer according to claim 1, further comprising a nucleic acid fragment having a nucleotide sequence represented by CACGTG, wherein the nucleic acid fragment is operably linked to the polypeptide (i) or (ii).

11. The enhancer according to claim 10, wherein the enhancer comprises one to ten of the nucleic acid fragments.

12. The enhancer according to claim 10, wherein the nucleic acid fragment consists of from 6 to 14 nucleotides.

13. A nucleic acid construct comprising the enhancer according to claim 1, and a promoter.

14. The nucleic acid construct according to claim 13, wherein the enhancer is operably inserted into, or operably linked to, the promoter.

15. A vector comprising the enhancer according to claim 1.

16. A vector comprising the nucleic acid construct according to claim 13.

17. A host cell comprising the enhancer according to claim 1.

18. A plant transfected with the enhancer according to claim 1.

19. A method for enhancing the transcription activity of a promoter, the method comprising a step of operably inserting the enhancer according to claim 1 into the promoter, or operably linking said enhancer to the promoter.

20. A method for increasing gene expression, the method comprising the steps of:
operably inserting the enhancer according to claim 1 into the promoter, or operably linking said enhancer to the promoter; and
causing expression of a gene located downstream of the promoter.

21. A method for creating a highly productive plant, the method comprising the steps of:
incorporating a nucleic acid molecule in a plant cell, wherein the nucleic acid molecule comprises the enhancer according to claim 1, and a promoter into which said enhancer is operably inserted or to which said enhancer is operably linked, and has a gene located downstream of the promoter; and
creating a plant from the plant cell incorporating the nucleic acid molecule.
